(19) United States
(12) Patent Application Publication
(10) Pub. No.: US 2018/0161427 A1
(43) Pub. Date: Jun. 14, 2018

(54) COMBINATION OF AN ANTI-IL-10 ANTIBODY AND A CPG-C TYPE OLIGONUCLEOTIDE FOR TREATING CANCER

(71) Applicants: Ying YU, Palo Alto, CA (US); Elliot Keith CHARTASH, Rahway, NJ (US); Svetlana SADEKOVA, Palo Alto, CA (US); Uyen Truong PHAN, Palo Alto, CA (US); Robert A. KASTELEIN, Palo Alto, CA (US); Robert L. COFFMAN, Berkeley, CA (US); Cristiana GUIDUCCI, Berkeley, CA (US); Robert S. JANSSEN, Berkeley, CA (US); MERCK SHARP & DOHME CORP., Rahway, NJ (US); DYNAVAX TECHNOLOGIES CORPORATION, Berkeley, CA (US)

(72) Inventors: Ying YU, Palo Alto, CA (US); Elliot Keith CHARTASH, Rahway, NJ (US); Svetlana SADEKOVA, Palo Alto, CA (US); Uyen Truong PHAN, Palo Alto, CA (US); Robert A. KASTELEIN, Palo Alto, CA (US); Robert L. COFFMAN, Berkeley, CA (US); Cristiana GUIDUCCI, Berkeley, CA (US); Robert S. JANSSEN, Berkeley, CA (US)

(73) Assignees: MERCK SHARP & DOHME CORP., Rahway, NJ (US); DYNAVAX TECHNOLOGIES CORPORATION, Berkeley, CA (US)

(21) Appl. No.: 15/577,377

(22) PCT Filed: May 26, 2016
(86) PCT No.: PCT/US16/34285

§ 371 Date: Nov. 28, 2017

Related U.S. Application Data
(60) Provisional application No. 62/168,470, filed on May 29, 2015.
(60) Provisional application No. 62/169,321, filed on Jun. 1, 2015.

(51) Int. Cl. A61K 39/395; A61K 31/7125; A61P 35/00

(52) U.S. Cl. CPC A61K 39/3955; A61K 2039/505; A61P 35/00; A61K 31/7125

(57) ABSTRACT

The present disclosure describes combination therapies comprising an anti-IL-10 antibody or antigen-binding fragment thereof and a CpG-C type oligonucleotide, and the use of the combination therapies for the treatment of cancer.

### FIELD OF THE INVENTION

The present invention relates to combination therapies useful for the treatment of cancer. In particular, the invention relates to a combination therapy which comprises an anti-IL-10 antibody and a TLR9 agonist that is a CpG-C type oligonucleotide.

### BACKGROUND OF THE INVENTION

Initially known as cytokine synthesis inhibitor factor or CSIF, interleukin-10 (IL-10) is a potent immunomodulator of hematopoietic cells, particularly immune cells. Cells such as activated Th2 cells, B cells, keratinocytes, monocytes and macrophages produce IL-10. See, e.g., Moore et al., Annu. Rev. Immunol. 11:165 (1993). IL-10 inhibits activation and effector functions of a number of cells that include T cells, monocytes and macrophages. In particular, IL-10 inhibits cytokine synthesis, including that of IL-1, IFN-γ, and TNF, by cells such as Th1 cells, natural killer cells, monocytes, and macrophages. See, e.g., Fiorentino et al., J. Exp. Med., 170:2081-2095 (1989); Fiorentino et al., J. Immunol. 146:3444 (1991); Hsu et al., Int. Immunol. 4:563 (1992); Hsu et al., Int. Immunol. 4:563 (1992); D'Andrea et al., J. Exp. Med. 178:1041 (1993); de Waal Malefyt et al., J. Exp. Med. 174:915 (1991); Fiorentino et al., J. Immunol. 147:3815 (1991).

The production of IL-10 in the tumor microenvironment by tumor infiltrating macrophages, dendritic cells, and CD4⁺ and CD8⁺ T cells has been shown to inhibit tumor eradication by the immune system (see, e.g., Jarnicki, et al. (2006)J. Immunol. 896-904). Targeting IL-10 with an antagonist of IL-10 could provide potent immunostimulatory activity and tumor eradication.

Administration of certain DNA sequences, generally known as immunostimulatory sequences, induces an immune response with a Th1-type bias as indicated by secretion of Th1-associated cytokines. Administration of an immunostimulatory polynucleotide with an antigen results in a Th1-type immune response to the administered antigen. Roman et al. (1997) Nature Med. 3:849-854. For example, mice injected intradermally with Escherichia coli (E. coli) β-galactosidase (β-Gal) in saline or in the adjuvant alum responded by producing specific IgG1 and IgE antibodies, and CD4⁺ cells that secreted IL-4 and IL-5, but not IFN-γ, demonstrating that the T cells were predominantly of the Th2 subset. However, mice injected intradermally (or with a tyne skin scratch applicator) with plasmid DNA (in saline) encoding β-Gal and containing an immunostimulatory sequence responded by producing IgG2a antibodies and CD4⁺ cells that secreted IFN-γ, but not IL-4 and IL-5, demonstrating that the T cells were predominantly of the Th1 subset. Moreover, specific IgE production by the plasmid DNA-injected mice was reduced 66-75%. Raz et al. (1996) Proc. Natl. Acad. Sci. USA 93:5141-5145. In general, the response to naked DNA immunization is characterized by production of IL-2, TNFα and IFN-γ by antigen-stimulated CD4⁺ T cells, which is indicative of a Th1-type response. This is particularly important in treatment of allergy and asthma as shown by the decreased IgE production. The ability of immunostimulatory polynucleotides to stimulate a Th1-type immune response has been demonstrated with bacterial antigens, viral antigens and with allergens (see, for example, WO 98/55495). There is a need in the art to improve the efficacy of cancer immunotherapy.

### SUMMARY OF THE INVENTION

In one embodiment, the invention provides a method for treating cancer in an individual comprising administering to the individual a combination therapy which comprises an anti-IL-10 antibody and a TLR9 agonist, wherein the TLR9 agonist is a CpG-C type oligonucleotide.

In another embodiment, the invention provides a medicament comprising an anti-IL-10 antibody for use in combination with a TLR9 agonist for treating cancer, wherein the TLR9 agonist is a CpG-C type oligonucleotide. In yet another embodiment, the invention provides a medicament comprising a TLR9 agonist for use in combination with an anti-IL-10 antibody for treating cancer, wherein the TLR9 agonist is a CpG-C type oligonucleotide.

Other embodiments provide use of an anti-IL-10 antibody in the manufacture of a medicament for treating cancer in an individual when administered in combination with a TLR9 agonist and use of a TLR9 agonist in the manufacture of a medicament for treating cancer in an individual when administered in combination with an anti-IL-10 antibody. In such embodiments, the TLR9 agonist is a CpG-C type oligonucleotide.

In a still further embodiment, the invention provides use of an anti-IL-10 antibody and a TLR9 agonist in the manufacture of medicaments for treating cancer in an individual, wherein the TLR9 agonist is a CpG-C type oligonucleotide. In some embodiments, the medicaments comprise a kit, and the kit also comprises a package insert comprising instructions for using the anti-IL-10 antibody in combination with the TLR9 agonist to treat cancer in an individual.

### BRIEF DESCRIPTION OF THE DRAWINGS

FIG. 1 shows amino acid sequences of anti-IL-10 hum12G8, with light chain sequence of SEQ ID NO: 2 and heavy chain sequence of SEQ ID NO: 1. The CDR regions are underlined.

FIG. 2 shows amino acid sequences of anti-IL-10 TC40.11D8, with light chain variable region sequence of SEQ ID NO: 3 and heavy chain variable region sequence of SEQ ID NO: 4.

FIG. 3 shows tumor growth of injected tumors in mouse TC-1 bilateral tumor model. Panel A shows volume of injected tumors for individual animals and number of complete regressions (CRs) per group. Panel B shows median volume of injected tumors with error bar indicating 68% confidence interval. Panel C compares volumes of injected tumors between treatment groups by day. Panel D shows unadjusted and multiplicity-adjusted P-values for comparison of volumes of injected tumors between treatments. Unadjusted p value refers to two-sided p-values based on the Peto & Peto version of the Gehan-Breslow nonparametric test statistic for right-censored data. P-values were estimated from 20,000 random reassignments of animals between the two treatments being compared. Multiplicity adjusted p-values refers to p-values adjusted to control the familywise error rate across all time points for a given pair of treatments. Adjustment was by applying the maxT procedure of Westfall and Young to the permutation distributions.

FIG. 4 shows tumor growth of non-injected tumors in mouse TC-1 bilateral tumor model. Panel A shows volume of non-injected tumors for individual animals and number of complete regressions (CRs) per group. Panel B shows median volume of non-injected tumors with error bar indicating 68% confidence interval. Panel C compares volumes of non-injected tumors between treatment groups by day. Panel D shows unadjusted and multiplicity-adjusted P-values for comparison of volumes of non-injected tumors between treatments. Unadjusted p value refers to two-sided p-values based on the Peto & Peto version of the Gehan-Breslow nonparametric test statistic for right-censored data. P-values were estimated from 20,000 random reassignments of animals between the two treatments being compared. Multiplicity adjusted p-values refers to p-values adjusted to control the familywise error rate across all time points for a given pair of treatments. Adjustment was by applying the maxT procedure of Westfall and Young to the permutation distributions.

FIG. 5 shows the induction of IFNα2a and IL-10 in human PBMCs (2 donors) with treatment of C59-08 and control ODN 1040 for 48 hours.

FIG. 6 shows induction of mRNA expression of IFNα-inducible genes (Panel A), cytokines (Panel B), and immune activation markers (Panel C) in a human renal cell carcinoma histoculture following treatment with C59-08 for 24 hours.

### DETAILED DESCRIPTION

Abbreviations.

Throughout the detailed description and examples of the invention the following abbreviations will be used:

BOR Best overall response

BID One dose twice daily

CBR Clinical Benefit Rate

CDR Complementarity determining region

CHO Chinese hamster ovary

CR Complete Response

DCR Disease Control Rate

DFS Disease free survival

DLT Dose limiting toxicity

DOR Duration of Response

DSDR Durable Stable Disease Rate

FFPE Formalin-fixed, paraffin-embedded

FR Framework region

IgG Immunoglobulin G

IHC Immunohistochemistry or immunohistochemical

irRC Immune related response criteria

IV Intravenous

MTD Maximum tolerated dose

NCBI National Center for Biotechnology Information

NCI National Cancer Institute

ORR Objective response rate

OS Overall survival

PD Progressive disease

PFS Progression free survival

PR Partial response

Q2W One dose every two weeks

Q3W One dose every three weeks

QD One dose per day

RECIST Response Evaluation Criteria in Solid Tumors

SD Stable disease

VH Immunoglobulin heavy chain variable region

VK Immunoglobulin kappa light chain variable region

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Administration”, as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. The term “subject” includes any organism, preferably an animal, more preferably a mammal (e.g., rat, mouse, dog, cat, rabbit) and most preferably a human.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab)₂, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Anti-tumor response” when referring to a cancer patient treated with a therapeutic regimen, such as a combination therapy described herein, means at least one positive therapeutic effect, such as for example, reduced number of cancer cells, reduced tumor size, reduced rate of cancer cell infiltration into peripheral organs, reduced rate of tumor metastasis or tumor growth, or progression free survival. Positive therapeutic effects in cancer can be measured in a number of ways (See, W. A. Weber, J. Null. Med. 50:1S-10S (2009); Eisenhauer et al., supra). In some embodiments, an anti-tumor response to a combination therapy described herein is assessed using RECIST 1.1 criteria, bidimentional irRC or unidimensional irRC. In some embodiments, an anti-tumor response is any of SD, PR, CR, PFS, or DFS.

“Bidimensional irRC” refers to the set of criteria described in Wolchok J D, et al. Guidelines for the evaluation of immune therapy activity in solid tumors: immune-related response criteria. Clin Cancer Res. 2009; 15(23):7412-7420. These criteria utilize bidimensional tumor measurements of target lesions, which are obtained by multiplying the longest diameter and the longest perpendicular diameter (cm²) of each lesion.

“Biotherapeutic agent” means a biological molecule, such as an antibody or fusion protein, that blocks ligand/receptor signaling in any biological pathway that supports tumor maintenance and/or growth or suppresses the anti-tumor immune response. Classes of biotherapeutic agents include, but are not limited to, antibodies to VEGF, EGFR, Her2/neu, other growth factor receptors, CD20, CD40, CD-40L, CTLA-4, OX-40, 4-1BB, and ICOS.

The terms “cancer”, “cancerous”, or “malignant” refer to or describe the physiological condition in mammals that is typically characterized by unregulated cell growth. Examples of cancer include but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre tumor cervical dysplasia), ovaries (ovarian carcinoma [serous cystadenocarcinoma, mucinous cystadenocarcinoma, unclassified carcinoma], granulosa thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. In another embodiment, the cancer is carcinoma, lymphoma, leukemia, blastoma, and sarcoma. More particular examples of such cancers include squamous cell carcinoma, myeloma, small-cell lung cancer, non-small cell lung cancer, glioma, hodgkin's lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Another particular example of cancer includes renal cell carcinoma. Yet another particular example of cancer is non-hodgkin's lymphoma or cutaneous T-cell lymphoma. Yet another particular example of cancer is acute myeloid leukemia (AML) or myelodysplastic syndrome.

“CpG-C ODNs” or “CpG-C type oligonucleotides” are oligonucleotides from 12 to 100 bases in length, which have one or more 5′-TCG trinucleotides wherein the 5′-T is positioned 0, 1, 2, or 3 bases from the 5′-end of the oligonucleotide, and at least one palindromic sequence of at least 8 bases in length comprising one or more unmethylated CG dinucleotides. The one or more 5′-TCG trinucleotide sequence may be separated from the 5′-end of the palindromic sequence by 0, 1, or 2 bases or the palindromic sequence may contain all or part of the one or more 5′-TCG trinucleotide sequence. In one embodiment, the oligonucleotide is an oligodeoxynucleotide (ODN). In one embodiment, the oligonucleotide is a 2′-oligodeoxynucleotide. CpG-C ODNs have the ability to stimulate B cells, induce plasmacytoid dendritic cell (PDC) maturation and cause secretion of high levels of type I interferons (e.g., IFN-α, IFN-γ, etc.). In some embodiments, the CpG-C ODNs are 12 to 100 bases in length, preferably 12 to 50 bases in length, preferably 12 to 40 bases in length, or preferably 12-30 bases in length. In some embodiments, the ODN is at least (lower limit) 12, 13, 14, 15, 16, 17, 18, 19, 20, 21, 22, 23, 24, 25, 26, 27, 28, 29, 30, 32, 34, 36, 38, 40, 50, 60, 70, 80, or 90 bases in length. In some embodiments, the ODN is at most (upper limit) 100, 90, 80, 70, 60, 50, 49, 48, 47, 46, 45, 44, 43, 42, 41, 40, 39, 38, 37, 36, 35, 34, 33, 32, 31, or 30 bases in length. In some embodiments, the at least one palindromic sequence is 8 to 97 bases in length, preferably 8 to 50 bases in length, or preferably 8 to 32 bases in length. In some embodiments, the at least one palindromic sequence is at least (lower limit) 8, 10, 12, 14, 16, 18, 20, 22, 24, 26, 28, or 30 bases in length. In some embodiments, the at least one palindromic sequence is at most (upper limit) 50, 48, 46, 44, 42, 40, 38, 36, 34, 32, 30, 28, 26, 24, 22, 20, 18, 16, 14, 12 or 10 bases in length. In one embodiment, the oligonucleotide is an oligodeoxynucleotide. In one embodiment, one or more of the internucleotide linkages of the CpG-C ODN are modified linkages. In one embodiment, one or more of the internucleotide linkages of CpG-C ODN are phosphorothioate (PS) linkages. In one embodiment, all of the internucleotide linkages of CpG-C ODN are phosphorothioate (PS) linkages. A phosphorothioate backbone refers to all of the internucleotide linkages of CpG-C ODN being phosphorothioate (PS) linkages.

The CpG-C type ODNs and SEQ ID NOs: 13-26 discussed herein are in their pharmaceutically acceptable salt form unless otherwise indicated. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In one embodiment, the CpG-C type ODNs are in the ammonium, sodium, lithium, or potassium salt form. In one preferred embodiment, the CpG-C type ODNs are in the sodium salt form. The CpG-C ODN may be provided in a pharmaceutical solution comprising a pharmaceutically acceptable excipient. Alternatively, the CpG-C ODN may be provided as a lyophilized solid, which is subsequently reconsistituted in sterile water, saline or a pharmaceutically acceptable buffer before administration.

Pharmaceutically acceptable excipients of the present disclosure include for instance, solvents, bulking agents, buffering agents, tonicity adjusting agents, and preservatives (see, e.g., Pramanick et al., Pharma Times, 45:65-77, 2013). In some embodiments the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent). The pharmaceutical compositions of the present disclosure are suitable for parenteral administration.

In some embodiments, the pharmaceutical compositions comprise an aqueous vehicle as a solvent. Suitable vehicles include for instance sterile water, saline solution, phosphate buffered saline, and Ringer's solution. In some embodiments, the composition is isotonic.

The pharmaceutical compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 4 to 9. In some embodiments, the pH is greater than (lower limit) 4, 5, 6, 7 or 8. In some embodiments, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical compositions may comprise a preservative. Suitable preservatives include for instance antioxidants and antimicrobial agents. However, in preferred embodiments, the pharmaceutical composition is prepared under sterile conditions and is in a single use container, and thus does not necessitate inclusion of a preservative.

The term “palindromic sequence” or “palindrome” refers to a nucleic acid sequence that is an inverted repeat, e.g., ABCDD′C′B′A′, where the bases, e.g., A, and A′, B and B′, C and C′, D and D′, are capable of forming Watson-Crick base pairs. Such sequences may be single-stranded or may form double-stranded structures or may form hairpin loop structures under some conditions. For example, as used herein, “an 8 base palindrome” refers to a nucleic acid sequence in which the palindromic sequence is 8 bases in length, such as ABCDD′C′B′A′. A palindromic sequence may be part of an oligonucleotide that also contains non-palindromic sequences. An oligonucleotide may contain one or more palindromic sequence portions and one or more non-palindromic sequence portions. Alternatively, an oligonucleotide sequence may be entirely palindromic. In an oligonucleotide with more than one palindromic sequence portion, the palindromic sequence portions may or may not overlap with each other.

In one embodiment, the CpG-C ODNs of the present disclosure comprise:

(a) 5′-Nₓ(TCG(N_{q}))_{y}N_{w}(X₁X₂CGX₂′X₁′(CG)ₚ)_{z,}Nᵥ (SEQ ID NO:13) wherein N are nucleosides, x=0, 1, 2 or 3, y=1, 2, 3 or 4, w=0, 1 or 2, p=0 or 1, q=0, 1 or 2, v=0 to 89 and z=1 to 20, X₁ and X₁′ are self-complementary nucleosides, X₂ and X₂′ are self-complementary nucleosides, and wherein the 5′-T of the (TCG(N_{q}))_{y}, sequence is 0-3 bases from the 5′ end of the oligonucleotide; and
(b) a palindromic sequence at least 8 bases in length wherein the palindromic sequence comprises the first (X₁X₂CGX₂′X₁′) of the (X₁X₂CGX₂′X₁′(CG)ₚ)_{z} sequences, wherein the ODN is from 12 to 100 bases in length. In some embodiments, x=0, y=1, w=0, p=0 or 1, q=0, 1 or 2, v=0 to 20 and z=1, 2, 3 or 4. In some embodiments, X₁ and X₂ are each either A or T. In some embodiments, the palindromic sequence has a base composition of more than one-third As and Ts. In some embodiments, the CpG-C ODN comprises a sequence selected from the group consisting of SEQ ID NOs:16-26.

In some embodiments, the CpG-C ODNs of the present disclosure consist of TCGN(X₁X₂CGX₂′X₁′CG)_{z}Nᵥ (SEQ ID NO:14), wherein N are nucleosides, q=0, 1, 2, 3, 4, or 5, v=0 to 20, z=1 to 4, X₁ and X₁′ are self-complementary nucleosides, X₂ and X₂′ are self-complementary nucleosides, and wherein the ODN is at least 12 bases in length. In some embodiments, the CpG-C ODN consists of a sequence selected from the group consisting of SEQ ID NOs:16-26.

In some embodiments, the CpG-C ODNs of the present disclosure consist of 5′-TCGN_{q}TTCGAACGTTCGAACGTTNₛ-3′ (SEQ ID NO:15), wherein N are nucleosides, q=0, 1, 2, 3, 4, or 5, s=0 to 20, and wherein the ODN is at least 12 bases in length. In one embodiment, s=0, 1, 2, 3, 4, or 5. In some embodiments, the CpG-C ODN consists of a sequence selected from the group consisting of 5′-TCGTTCGAACGTTCGAACGTTCGAA-3′ (SEQ ID NO:17) q=0 and s=4, 5′-TCGAACGTTCGAACGTTCGAACGTT-3′ (SEQ ID NO:18) q=4 and s=0, 5′-TCGAACGTTCGAACGTTCGAACGTTCGAAT-3′ (SEQ ID NO:20) q=4 and s=5, 5′-TCGTAACGTTCGAACGTTCGAACGTTA-3′ (SEQ ID NO:21) q=5 and s=1, and 5′-TCGTAACGTTCGAACGTTCGAACGTT-3′ (SEQ ID NO:22) q=5 and s=0.

In one embodiment, the TLR9 agonist is a CpG-C ODN consisting of the sequence 5′-TCGAACGTTCGAACGTTCGAACGTTCGAAT-3′ (SEQ ID NO:20). In another embodiment, the CpG-C ODN is the sodium salt of 5′TCGAACGTTCGAACGTTCGAACGTTCGAAT-3′ (SEQ ID NO:20). In a further embodiment, the CpG-C type oligonucleotide has a sequence that consists of 5′-TCGTTCGAACGTTCGAACGTTCGAA-3′ (SEQ ID NO:17). In a further embodiment, the CpG-C type oligonucleotide is a sodium salt of 5′-TCGTTCGAACGTTCGAACGTTCGAA-3′ (SEQ ID NO:17).

In another embodiment, the TLR9 agonist CpG-C type oligonucleotide is selected from the group consisting of:

(SEQ ID NO: 16)
5′-TCGTCGAACGTTCGAGATGAT-3′;
(SEQ ID NO: 17)
5′-TCGTTCGAACGTTCGAACGTTCGAA-3′;
(SEQ ID NO: 18)
5′-TCGAACGTTCGAACGTTCGAACGTT-3′;
(SEQ ID NO: 19)
5′-TCGAACGTTCGAACGTTCGAATTTT-3′;
(SEQ ID NO: 20)
5′-TCGAACGTTCGAACGTTCGAACGTTCGAAT-3′;
(SEQ ID NO: 21)
5′-TCGTAACGTTCGAACGTTCGAACGTTA-3′ ;
(SEQ ID NO: 22)
5′-TCGTAACGTTCGAACGTTCGAACGTT-3′;
(SEQ ID NO: 23)
5′-TCGTAACGTTCGAACGTTCGAACGT-3′;
(SEQ ID NO: 24)
5′-TCGTAACGTTCGAACGTTCGAACG-3′;
(SEQ ID NO: 25)
5′-TCGTAACGTTCGAACGTTCGAAC-3′;
and
(SEQ ID NO: 26)
5′-TCGTAACGTTCGAACGTTCGAA-3′.

TABLE 1
Motif and Sequences of CpG-C type Oligonucleotides

| | SEQ | |
|---|---|---|
| Compound | ID | |
| # | NO: | Sequence |
| C59-01 | 13 | 5′-Nₓ(TCG(N_{q}))_{y}N_{w}(X₁X₂CGX₂′X₁′(CG)ₚ)_{z}Nᵥ- |
| | | 3′ |
| C59-02 | 14 | 5′-TCGN_{q}(X₁X₂CGX₂′X₁′CG)_{z}Nᵥ-3′ |
| C59-03 | 15 | 5′-TCGN_{q}TTCGAACGTTCGAACGTTN,-3′ |
| C59-04 | 16 | 5′-TCGTCGAACGTTCGAGATGAT-3′ |
| C59-05 | 17 | 5′-TCGTTCGAACGTTCGAACGTTCGAA-3′ |
| C59-06 | 18 | 5′-TCGAACGTTCGAACGTTCGAACGTT-3′ |
| C59-07 | 19 | 5′-TCGAACGTTCGAACGTTCGAATTTT-3′ |
| C59-08 | 20 | 5′-TCGAACGTTCGAACGTTCGAACGTTCGAAT-3′ |
| C59-09 | 21 | 5′-TCGTAACGTTCGAACGTTCGAACGTTA-3′ |
| C59-10 | 22 | 5′-TCGTAACGTTCGAACGTTCGAACGTT-3′ |
| C59-11 | 23 | 5′-TCGTAACGTTCGAACGTTCGAACGT-3′ |
| C59-12 | 24 | 5′-TCGTAACGTTCGAACGTTCGAACG-3′ |
| C59-13 | 25 | 5′-TCGTAACGTTCGAACGTTCGAAC-3′ |
| C59-14 | 26 | 5′-TCGTAACGTTCGAACGTTCGAA-3′ |

It is understood that, with respect to formulae or sequence motifs described herein, any and all parameters are independently selected. For example, if x=0-2, y may be independently selected regardless of the value of x (or any other selectable parameter in a formula), as long as the total oligonucleotide length limitation is met.

Additional CpG-C oligonucleotides having sequences encompassed by the motifs of the present disclosure are suitable for use in the methods and medicaments disclosed herein. A plurality of additional CpG-C oligonucleotides having sequences encompassed by the motifs of the present disclosure are described in U.S. Pat. Nos. 7,745,606, 8,158,768, and 8,871,732 to Dynavax Technologies Corporation. These sequences are hereby incorporated by reference.

CpG oligonucleotides have been described in the art and their activity may be readily determined using standard assays, which measure various aspects of immune responses (e.g., cytokine secretion, antibody production, NK cell activation, B cell proliferation, T cell proliferation, etc.). Exemplary methods are described in WO 97/28259; WO 98/16247; WO 99/11275, WO 98/55495 and WO 00/61151, as well as U.S. Pat. Nos. 7,745,606, 8,158,768, and 8,871,732 to Dynavax Technologies Corporation. Accordingly, these and other methods can be used to detect and quantify immunomodulatory activity of CpG oligonucleotides.

CpG-C oligonucleotides may contain modifications. Suitable modifications include but are not limited to, modifications of the 3′0H or 5′0H group, modifications of the nucleotide base, modifications of the sugar component, and modifications of the phosphate group. Modified bases may be included in the palindromic sequence as long as the modified base(s) maintains the same specificity for its natural complement through Watson-Crick base pairing (e.g., the palindromic portion of the CpG-C oligonucleotide remains self-complementary).

CpG-C oligonucleotides may be linear, may be circular or include circular portions and/or a hairpin loop. CpG-C oligonucleotides may be single stranded or double stranded. CpG-C oligonucleotides may be DNA, RNA or a DNA/RNA hybrid.

CpG-C oligonucleotides may contain naturally-occurring or modified, non-naturally occurring bases, and may contain modified sugar, phosphate, and/or termini. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. In some embodiments, CpG-C oligonucleotides have only phosphorothioate linkages, only phosphodiester linkages, or a combination of phosphodiester and phosphorothioate linkages.

Sugar modifications known in the field, such as 2′-alkoxy-RNA analogs, 2′-amino-RNA analogs, 2′-fluoro-DNA, and 2′-alkoxy- or amino-RNA/DNA chimeras and others described herein, may also be made and combined with any phosphate modification. Examples of base modifications include but are not limited to addition of an electron-withdrawing moiety to C-5 and/or C-6 of a cytosine of the CpG-C oligonucleotide (e.g., 5-bromocytosine, 5-chlorocytosine, 5-fluorocytosine, 5-iodocytosine) and C-5 and/or C-6 of a uracil of the CpG-C oligonucleotide (e.g., 5-bromouracil, 5-chlorouracil, 5-fluorouracil, 5-iodouracil). As noted above, use of a base modification in a palidromic sequence of a CpG-C oligonucleotide should not interfere with the self-complementarity of the bases involved for Watson-Crick base pairing. However, outside of a palindromic sequence, modified bases may be used without this restriction. For instance, 2′-O-methyl-uridine and 2′-O-methyl-cytidine may be used outside of the palindromic sequence, whereas, 5-bromo-2′-deoxycytidine may be used both inside and outside the palindromic sequence. Other modified nucleotides, which may be employed both inside and outside of the palindromic sequence include 7-deaza-8-aza-dG, 2-amino-dA, and 2-thio-dT.

Duplex (i.e., double stranded) and hairpin forms of most oligonucleotides are in dynamic equilibrium, with the hairpin form generally favored at low oligonucleotide concentration and higher temperatures. Covalent interstrand or intrastrand cross-links increase duplex or hairpin stability, respectively, towards thermal-, ionic-, pH-, and concentration-induced conformational changes. Chemical cross-links can be used to lock the polynucleotide into either the duplex or the hairpin form for physicochemical and biological characterization. Cross-linked oligonucleotides that are conformationally homogeneous and are “locked” in their most active form (either duplex or hairpin form) could potentially be more active than their uncross-linked counterparts. Accordingly, some CpG-C oligonucleotides of the present disclosure contain covalent interstrand and/or intrastrand cross-links.

A variety of ways to chemically cross-link duplex DNA are known in the art. Any cross-linking method may be used as long as the cross-linked polynucleotide product possesses the desired immunomodulatory activity. One method, for example, results in a disulfide bridge between two opposing thymidines at the terminus of the duplex or hairpin. For this cross-linking method, the oligonucleotide(s) of interest is synthesized with a 5′-DMT-N3-(tBu-SS-ethyl)thymidine-3′-phosphoramidite (“T*”). To form the disulfide bridge, the mixed disulfide bonds are reduced, oligonucleotide purified, the strands hybridized and the compound air-oxidized to form the intrastrand cross-link in the case of a hairpin form or the interstrand cross-link in the case of a duplex form. Alternatively, the oligonucleotides may be hybridized first and then reduced, purified and air-oxidized. Such methods and others are described in the art (see, e.g., Glick et al., J Org Chem, 56:6746-6747, 1991, Glick et al., J Am Chem Soc, 114:5447-5448, 1992, Goodwin et al., Tetrahedron Letters 35:1647-1650, 1994, Wang et al., J Am Chem Soc, 117:2981-2991, 1995, Osborne et al., Bioorganic & Medicinal Chemistry Letters, 6:2339-2342, 1996 and Osborne et al., J Am Chem Soc, 118:11993-12003, 1996).

Another cross-linking method forms a disulfide bridge between offset residues in the duplex or hairpin structure. For this cross-linking method, the oligonucleotide(s) of interest is synthesized with convertible nucleosides (commercially available, for example, from Glen Research). This method utilizes, for example, an A-A disulfide or a C-A disulfide bridge and linkages through other bases are also possible. To form the disulfide-modified polynucleotide, the polynucleotide containing the convertible nucleoside is reacted with cystamine (or other disulfide-containing amine). To form the disulfide bridge, the mixed disulfide bonds are reduced, oligonucleotide purified, the strands hybridized and the compound air-oxidized to form the intrastrand cross-link in the case of a hairpin form or the interstrand cross-link in the case of a duplex form. Alternatively, the oligonucleotides may be hybridized first and then reduced, purified and air-oxidized. Such methods are described in the art (see, e.g., Ferentz et al., J Am Chem Soc, 113:4000-4002, 1991, and Ferentz et al., J Am Chem Soc, 115:9006-9014, 1993).

The techniques for making polynucleotides and modified polynucleotides are known in the art. Naturally occurring DNA or RNA, containing phosphodiester linkages, is generally synthesized by sequentially coupling the appropriate nucleoside phosphoramidite to the 5′-hydroxy group of the growing oligonucleotide attached to a solid support at the 3′-end, followed by oxidation of the intermediate phosphite triester to a phosphate triester. Once the desired polynucleotide sequence has been synthesized, the polynucleotide is removed from the support, the phosphate triester groups are deprotected to phosphate diesters and the nucleoside bases are deprotected using aqueous ammonia or other bases (see, e.g., Beaucage “Oligodeoxyribonucleotide Synthesis” in Protocols for Oligonucleotides and Analogs, Synthesis and Properties (Agrawal, ed.) Humana Press, Totowa, N.J., 1993; Warner et al., DNA 3:401, 1984 and U.S. Pat. No. 4,458,066).

The CpG-C oligonucleotide may contain phosphate-modified oligonucleotides, some of which are known to stabilize the oligonucleotide. Accordingly, some embodiments include stabilized CpG-C oligonucleotides. Synthesis of oligonucleotides containing modified phosphate linkages or non-phosphate linkages is also known in the art (see, e.g., Matteucci “Oligonucleotide Analogs: an Overview” in Oligonucleotides as Therapeutic Agents, (D. J. Chadwick and G. Cardew, ed.) John Wiley and Sons, New York, N.Y., 1997). The phosphorous derivative (or modified phosphate group), which can be attached to the sugar or sugar analog moiety in the oligonucleotide, can be a monophosphate, diphosphate, triphosphate, alkylphosphonate, phosphorothioate, phosphorodithioate, phosphoramidate or the like. The preparation of the above-noted phosphate analogs, and their incorporation into nucleotides, modified nucleotides and oligonucleotides, per se, has already been well described (see, e.g., Peyrottes et al., Nucleic Acids Res, 24:1841-1848, 1996; Chaturvedi et al., Nucleic Acids Res, 24:2318-2323, 1996; and Schultz et al., Nucleic Acids Res, 24:2966-2973, 1996). For example, synthesis of phosphorothioate oligonucleotides is similar to that described above for naturally occurring oligonucleotides except that the oxidation step is replaced by a sulfurization step (Zon “Oligonucleoside Phosphorothioates” in Protocols for Oligonucleotides and Analogs, Synthesis and Properties (Agrawal, ed.) Humana Press, pp. 165-190, 1993).

CpG-C oligonucleotides can comprise one or more ribonucleotides (containing ribose as the only or principal sugar component), deoxyribonucleotides (containing deoxyribose as the principal sugar component), modified sugars or sugar analogs. Thus, in addition to ribose and deoxyribose, the sugar moiety can be pentose, deoxypentose, hexose, deoxyhexose, glucose, arabinose, xylose, lyxose, and a sugar analog cyclopentyl group. The sugar can be in pyranosyl or in a furanosyl form. In the CpG-C oligonucleotide, the sugar moiety is preferably the furanoside of ribose, deoxyribose, arabinose or 2′-0-alkylribose, and the sugar can be attached to the respective heterocyclic bases either in anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification in the CpG-C oligonucleotide includes, but is not limited to, 2′-O-methyl-uridine and 2′-O-methyl-cytidine. The preparation of these sugars or sugar analogs and the respective nucleosides wherein such sugars or analogs are attached to a heterocyclic base (nucleic acid base) per se is known, and therefore need not be described here. Sugar modifications may also be made and combined with any phosphate modification in the preparation of a CpG-C oligonucleotide.

The heterocyclic bases, or nucleic acid bases, which are incorporated in the CpG-C oligonucleotide can be the naturally-occurring principal purine and pyrimidine bases, (namely uracil, thymine, cytosine, adenine and guanine, as mentioned above), as well as naturally-occurring and synthetic modifications of said principal bases. Thus, a CpG-C oligonucleotide may include one or more of inosine, 2′-deoxyuridine, and 2-amino-2′-deoxyadenosine.

“CBR” or “Clinical Benefit Rate” means CR+PR+durable SD.

“CDR” or “CDRs” as used herein means complementarity determining region(s) in a immunoglobulin variable region, defined using the Kabat numbering system, unless otherwise indicated.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods of the present invention include cytostatic and/or cytotoxic agents.

“Chothia” as used herein means an antibody numbering system described in Al-Lazikani et al., JMB 273:927-948 (1997).

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, an anti-IL-10 antibody that consists essentially of a recited amino acid sequence may also include one or more amino acids, including substitutions of one or more amino acid residues, which do not materially affect the properties of the binding compound.

“DCR” or “Disease Control Rate” means CR+PR+SD.

“DSDR” or “Durable Stable Disease Rate” means SD for >23 weeks.

“Framework region” or “FR” as used herein means the immunoglobulin variable regions excluding the CDR regions.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991)J Mol. Biol. 222: 581-597, for example. See also Presta (2005) J Allergy Clin. Immunol. 116:731.

“Non-responder patient”, when referring to a specific anti-tumor response to treatment with a combination therapy described herein, means the patient did not exhibit the anti-tumor response.

“ORR” or “objective response rate” refers in some embodiments to CR+PR, and ORR_{(week 24)} refers to CR and PR measured using irRECIST in each patient in a cohort after 24 weeks of treatment with the combinations of the invention.

“Patient” or “subject” refers to any single subject for which therapy is desired or that is participating in a clinical trial, epidemiological study or used as a control, including humans and mammalian veterinary patients such as cattle, horses, dogs, and cats.

“Anti-IL-10 antibody” means an antagonist antibody that binds IL-10 to inhibit the activity of IL-10. Alternative names or synonyms for IL-10 include: Interleukin-10, cytokine synthesis inhibitor factor or CSIF. Human IL-10 amino acid sequences can be found in U.S. Pat. No. 6,217,857. The amino acid sequence of the mature human IL-10 protein is SPGQGTQSENSCTHFPGNLPNMLRDLRDAF SRVKTFFQMKDQLDNLLLKESLLEDFKGY LGCQALSEMIQFYLEEVMPQAENQDPDIKAHVNSLGENLKTLRLRLRRCHRFLPCENKS KAVEQVKNAFNKLQEKGIYKAMSEFDIFINYIEAYMTMKIRN (SEQ ID NO: 28)

Anti-IL-10 antibodies useful in any of the treatment method, medicaments and uses of the present invention include a monoclonal antibody (mAb), or antigen binding fragment thereof, which specifically binds to IL-10. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

In some preferred embodiments of the treatment method, medicaments and uses of the present invention, the anti-IL-10 antibody is a monoclonal antibody, or antigen binding fragment thereof, which comprises: (a) light chain CDRs of SEQ ID NOs: 5, 6 and 7 and heavy chain CDRs SEQ ID NOs: 8, 9 and 10 of anti-IL-10 hum12G8. In other preferred embodiments of the treatment method, medicaments and uses of the present invention, the anti-IL-10 antibody is a monoclonal antibody, or antigen binding fragment thereof, which comprises: (a) light chain CDRs of SEQ ID NOs: 31, 32 and 33 and heavy chain CDRs SEQ ID NOs: 34, 35 and 36 of anti-IL-10 hum11D8.

In other preferred embodiments of the treatment method, medicaments and uses of the present invention, the anti-IL-10 antibody is a monoclonal antibody, or antigen binding fragment thereof, which specifically binds to human IL-10 and comprises (a) a heavy chain variable region comprising SEQ ID NO:11 or a variant thereof, and (b) a light chain variable region comprising an amino acid sequence of SEQ ID NO:12 or a variant thereof. In yet other preferred embodiments of the treatment method, medicaments and uses of the present invention, the anti-IL-10 antibody is a monoclonal antibody, or antigen binding fragment thereof, which specifically binds to human IL-10 and comprises (a) a heavy chain variable region comprising SEQ ID NO:4 or a variant thereof, and (b) a light chain variable region comprising an amino acid sequence of SEQ ID NO:3 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

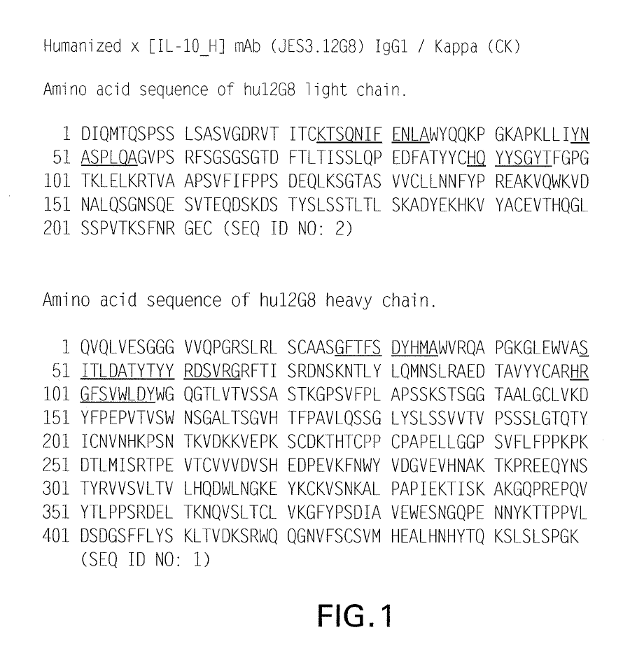

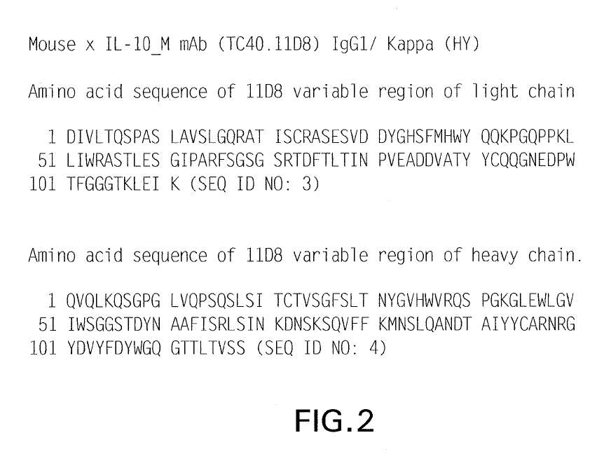

Table 3 below provides a list of the amino acid sequences of exemplary anti-IL-10 mAbs for use in the treatment method, medicaments and uses of the present invention, and the sequences are shown in FIGS. 1-2.

TABLE 3
EXEMPLARY ANTI-HUMAN IL-10 MONOCLONAL ANTIBODIES
A. Comprises light and heavy chain CDRs of hum12G8 in US patent 7662379

| | |
|---|---|
| CDRL1 | SEQ ID NO: 5 KTSQNIFENLA |
| CDRL2 | SEQ ID NO: 6 YNASPLQA |
| CDRL3 | SEQ ID NO: 7 HQYYSGYT |
| CDRH1 | SEQ ID NO: 8 GFTFSDYHMA |
| CDRH2 | SEQ ID NO: 9 SITLDATYTYYRDSVRG |
| CDRH3 | SEQ ID NO: 10 HRGFSVWLDY |

B. Comprises the heavy chain variable region and light chain variable regions of
hum12G8 in U.S. Pat. No. 7662379

| | |
|---|---|
| Heavy chain VR | SEQ ID NO: 11 |
| | QVQLVESGGGVVQPGRSLRLSCAASGFTFSDYHMAWVRQAPGKGLEWVA |
| | S |
| | ITLDATYTYYRDSVRGRFTISRDNSKNTLYLQMNSLRAEDTAVYYCARHR |
| | GFSVWLDYWGQGTLVTVSSA |
| Light chain VR | SEQ ID NO: 12 |
| | DIQMTQSPSSLSASVGDRVTITCKTSQNIFENLAWYQQKPGKAPKLLIYN |
| | ASPLQAGVPSRFSGSGSGTDFTLTISSLQPEDFATYYCHQYYSGYTFGPG |
| | TKLELKRTVAA |

C. Comprises the heavy chain and light chain of hum12G8 in U.S. Pat. No. 7662379

| | |
|---|---|
| Heavy chain | SEQ ID NO: 1 |
| Light chain | SEQ ID NO: 2 |

D. Comprises the heavy chain and light chain of 11D8 in U.S. Pat. No. 8226947

| | |
|---|---|
| Heavy chain | SEQ ID NO: 29 |
| | QVQLVESGGGVVQPGRSLRLSCAASGFSLTNYGVHWVRQAPGKGLEWVA |
| | VIWSGGSTDYNAAFISRFTISRDNSKNTLYLQMNSLRAEDTAVYYCARNRG |
| | YDVYFDYWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| Light chain | SEQ ID NO: 30 |
| | EIVLTQSPGTLSLSPGERATLSCRASESVDDYGHSFMHWYQQKPGQAPRLLI |
| | YRASTLESGIPDRFSGSGSGTDFTLTISRLEPEDFAVYYCQQGNEDPWTFGQ |
| | GTKVEIKRTVAAPSVFIFPPSDEQLKSGTASVVCLLNNFYPREAKVQWKVD |
| | NALQSGNSQESVTEQDSKDSTYSLSSTLTLSKADYEKHKVYACEVTHQGLS |
| | SPVTKSFNRGEC |

E. Comprises light and heavy chain CDRs of hum11D8 in U.S. Pat. No. 8226947 and of TC40.11D8

| | |
|---|---|
| CDRL1 | SEQ ID NO: 31: RASESVDDYGHSFMH |
| CDRL2 | SEQ ID NO: 32: RASTLES |
| CDRL3 | SEQ ID NO: 33: QQGNEDPWT |
| CDRH1 | SEQ ID NO: 34: GFSLTNYGVH |
| CDRH2 | SEQ ID NO: 35: VIWSGGSTDYNAAFIS |
| CDRH3 | SEQ ID NO: 36: NRGYDVYFDY |

F: Comprises light and heavy chain variable regions of TC40.11D8

| | |
|---|---|
| Light chain | SEQ ID NO: 3 |
| Heavy chain | SEQ ID NO: 4 |

As used herein, an “anti-IL-10 hum 12G8 variant” means a monoclonal antibody which comprises heavy chain and light chain sequences that are identical to those in anti-IL-10 hum 12G8, except for having three, two or one conservative amino acid substitutions at positions that are located outside of the light chain CDRs and six, five, four, three, two or one conservative amino acid substitutions that are located outside of the heavy chain CDRs, e.g, the variant positions are located in the FR regions or the constant region. In other words, anti-IL-10 hum 12G8 and an anti-IL-10 hum 12G8 variant comprise identical CDR sequences, but differ from each other due to having a conservative amino acid substitution at no more than three or six other positions in their full length light and heavy chain sequences, respectively. An anti-IL-10 hum 12G8 variant is substantially the same as anti-IL-10 hum 12G8 with respect to the following properties: binding affinity to IL-10 and neutralizing effect in vivo.

“RECIST 1.1 Response Criteria” as used herein means the definitions set forth in Eisenhauer et al., E. A. et al., Eur. J Cancer 45:228-247 (2009) for target lesions or nontarget lesions, as appropriate based on the context in which response is being measured.

“Responder patient” when referring to a specific anti-tumor response to treatment with a combination therapy described herein, means the patient exhibited the anti-tumor response.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Tissue Section” refers to a single part or piece of a tissue sample, e.g., a thin slice of tissue cut from a sample of a normal tissue or of a tumor.

“Treat” or “treating” cancer as used herein means to administer a combination therapy of an anti-IL-10 antibody and CpG-C type oligonucleotide to a subject having cancer, or diagnosed with cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, response to a combination therapy described herein is assessed using RECIST 1.1 criteria or irRC (bidimensional or unidimensional) and the treatment achieved by a combination of the invention is any of PR, CR, OR, PFS, DFS and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination of the invention is any of PR, CR, PFS, DFS, OR and OS that is assessed using RECIST 1.1 response criteria. The treatment regimen for a combination of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as the Student's t-test, the chi^{t}-test, the U-test according to Mann and Whitney, the Kruskal-Wallis test (H-test), Jonckheere-Terpstra-test and the Wilcoxon-test.

The terms “treatment regimen”, “dosing protocol” and “dosing regimen” are used interchangeably to refer to the dose and timing of administration of each therapeutic agent in a combination of the invention.

“Tumor” as it applies to a subject diagnosed with, or suspected of having, cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone marrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MM) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Unidimensional irRC refers to the set of criteria described in Nishino M, Giobbie-Hurder A, Gargano M, Suda M, Ramaiya N H, Hodi F S. Developing a Common Language for Tumor Response to Immunotherapy: Immune-related Response Criteria using Unidimensional measurements. Clin Cancer Res. 2013; 19(14):3936-3943). These criteria utilize the longest diameter (cm) of each lesion.

“Variable regions” or “V region” as used herein means the segment of IgG chains which is variable in sequence between different antibodies. It extends to Kabat residue 109 in the light chain and 113 in the heavy chain.

Any IL-10 antibody could be used in the combinations of the invention. In one embodiment, the anti-IL-10 antibodies to be used are the ones described in U.S. Pat. No. 8,226,947 and U.S. Pat. No. 7,662,379, the disclosure of which is hereby incorporated by reference in its entirety. In another embodiment, the anti-IL-10 antibody is anti-IL-10 hum12G8, which comprises two identical light chains with the sequence of SEQ ID NO: 2 and two identical heavy chains with the sequence of SEQ ID NO: 1. Plasmids containing nucleic acids encoding both the heavy and light chains of hum12G8 were deposited with the ATCC on May 6, 2004, as PTA-5922 and PTA-5923, respectively. In a further embodiment, the anti-IL-10 antibody are those described in U.S. Patent Publication No. US2012/0321617 (humanized hVH20/hVL7, hVH20/hVL8, hVH26/hVL7 and chimeric cB-N10)).

### II. Methods, Uses and Medicaments

In one aspect of the invention, the invention provides a method for treating cancer in an individual comprising administering to the individual a combination therapy which comprises an anti-IL-10 antibody and a CpG-C type oligonucleotide.

The combination therapy may also comprise one or more additional therapeutic agents. The additional therapeutic agent may be, e.g., a chemotherapeutic other than a CpG-C type oligonucleotide, a biotherapeutic agent, immunotherapeutic agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), cells transfected with genes encoding immune stimulating cytokines such as but not limited to GM-CSF), and radiation. In some embodiments, the immunotherpaeutic agent comprises one or more of a cytokine, a small molecule adjuvant, and an antibody. In some embodiments, the cytokine comprises one or more of a chemokine, an interferon, an interleukin, a lymphokine, and a tumour necrosis factor. The specific dosage and dosage schedule of the additional therapeutic agent can further vary, and the optimal dose, dosing schedule and route of administration will be determined based upon the specific therapeutic agent that is being used.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammall and calicheamicin phiI1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a combination therapy of the invention may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, the CpG-C type oligonucleotide is administered before administration of the anti-IL-10 antibody, while in other embodiments, the CpG-C type oligonucleotide is administered after administration of the anti-IL-10 antibody. In another embodiment, the CpG-C type oligonucleotide is administered concurrently with the anti-IL-10 antibody.

In some embodiments, the CpG-C type oligonucleotide is administered intratumorally or intravenously. In another embodiment, the anti-IL-10 antibody is administered intratumorally or intravenously. In another embodiment, the CpG-C type oligonucleotide is administered intratumorally and the anti-IL-10 antibody is administered intravenously.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

Each small molecule therapeutic agent in a combination therapy of the invention can be administered orally or parenterally, including the intravenous, intramuscular, intraperitoneal, subcutaneous, rectal, topical, and transdermal routes of administration.

A combination therapy of the invention may be used prior to or following surgery to remove a tumor and may be used prior to, during or after radiation therapy.

In some embodiments, a combination therapy of the invention is administered to a patient who has not been previously treated with a biotherapeutic or chemotherapeutic agent, i.e., is treatment-naive. In other embodiments, the combination therapy is administered to a patient who failed to achieve a sustained response after prior therapy with a biotherapeutic or chemotherapeutic agent, i.e., is treatment-experienced.

A combination therapy of the invention is typically used to treat a tumor that is large enough to be found by palpation or by imaging techniques well known in the art, such as Mill, ultrasound, or CAT scan.

Selecting a dosage regimen (also referred to herein as an administration regimen) for a combination therapy of the invention depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, on the patient's clinical history (e.g., previous therapy), the type and stage of the cancer to be treated and biomarkers of response to one or more of the therapeutic agents in the combination therapy.

Biotherapeutic agents in a combination therapy of the invention may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144.

In one embodiment of the invention, the anti-IL-10 antibody in the combination therapy is anti-IL-10 hum 12G8, which is administered intravenously at a dose selected from the group consisting of: 1 mg/kg Q3W, 2 mg/kg Q3W, 3 mg/kg Q3W, 4 mg/kg Q3W, 5 mg/kg Q3W, 6 mg/kg Q3W, 7 mg/kg Q3W, 8 mg/kg Q3W, 9 mg/kg Q3W, 10 mg/kg Q3W, 11 mg/kg Q3W, 12 mg/kg Q3W, 13 mg/kg Q3W, 14 mg/kg Q3W and 15 mg/kg Q3W. In another embodiment of the invention, the anti-IL-10 antibody in the combination therapy is anti-IL-10 hum 12G8, which is administered intravenously at a dose of 1 mg/kg Q3W. In a further embodiment of the invention, the anti-IL-10 antibody in the combination therapy is anti-IL-10 hum 12G8, which is administered intravenously at a dose of 3 mg/kg Q3W. In yet another embodiment of the invention, the anti-IL-10 antibody in the combination therapy is anti-IL-10 hum 12G8, which is administered intravenously at a dose of 10 mg/kg Q3W. In other embodiments of the invention, the anti-IL-10 antibody in the combination therapy is anti-IL-10 hum 12G8, which is administered intravenously on Day 1 at a dose of 70 mg, 210 mg or 700 mg Q3W, optionally for 7 additional doses.

In a preferred embodiment of the invention, the anti-IL-10 antibody in the combination therapy is anti-IL-10 hum 12G8, or an anti-IL-10 hum 12G8 variant, which is administered in a liquid medicament at a dose selected from the group consisting of 1 mg/kg Q3W, 2 mg/kg Q3W, 3 mg/kg Q3W, 4 mg/kg Q3W, 5 mg/kg Q3W, 6 mg/kg Q3W, 7 mg/kg Q3W, 8 mg/kg Q3W, 9 mg/kg Q3W, 10 mg/kg Q3W, 11 mg/kg Q3W, 12 mg/kg Q3W, 13 mg/kg Q3W, 14 mg/kg Q3W and 15 mg/kg Q3W.

In one embodiment of the invention, the CpG-C type oligonucleotide in the combination therapy is an oligonucleotide of SEQ ID NO:20, which is administered intratumorally at a dose selected from the group consisting of 0.1, 0.5, 1.0, 2.0, 3.0, 4.0, 5.0, 6.0, 7.0, 8.0 or 16.0 mg, or from 0.1-16 mg. In some embodiments, the CpG-C type oligonucleotide of SEQ ID NO:20 is administered twice weekly, once weekly, biweekly, once every three weeks, once a month, or bimonthly. In another embodiment of the invention, the CpG-C type oligonucleotide of SEQ ID NO:20 is administered intratumorally at a dose selected from the group consisting of 0.1, 0.5, 1.0, 2.0, 3.0, 4.0, 5.0, 6.0, 7.0, 8.0 or 16.0 mg, or from 0.1-16 mg weekly for four times. In another embodiment of the invention, the CpG-C type oligonucleotide of SEQ ID NO:20 is administered intratumorally at a dose selected from the group consisting of 0.1, 0.5, 1.0, 2.0, 3.0, 4.0, 5.0, 6.0, 7.0, 8.0 or 16.0 mg on Days 1 and 8 or Days 1, 8, 15 and 22. In yet another embodiment of the invention, the CpG-C type oligonucleotide of SEQ ID NO:20 is administered intratumorally at a dose selected from the group consisting of 2.0, 4.0 or 8.0 mg on Days 1, 8, 15, 22, 43, 50, 57 and 64. In other embodiments of the invention, the CpG-C type oligonucleotide of SEQ ID NO:20 is administered intratumorally at a dose of 1.0 or 4.0 mg on Days 1, 8, 15, 22, then Q3W, optionally for 6 additional doses.

CpG-C type oligonucleotide can be administered in accordance with any dose and dosing schedule that, together with the effect of the anti-IL-10 antibody, achieves a dose effective to treat cancer. The optimal dose for the anti-IL-10 antibody in combination with a CpG-C type oligonucleotide may be identified by dose escalation or dose de-escalation of one or both of these agents. In an embodiment, the combination therapy comprises 1-10 mg/kg intravenous infusion of anti-IL-10 hum 12G8 once every three weeks and intratumoral administration of 1-16 mg of the CpG-C type oligonucleotide of SEQ ID NO:20 weekly. In another embodiment, the combination therapy comprises 1 or 3 mg/kg intravenous infusion of anti-IL-10 hum 12G8 once every three weeks and intratumoral administration of 2, 4 or 8 mg of the CpG-C type oligonucleotide of SEQ ID NO:20 thereof weekly. In a further embodiment, the combination therapy comprises 10 mg/kg intravenous infusion of anti-IL-10 hum 12G8 once every three weeks and intratumoral administration of 2, 4 or 8 mg of the CpG-C type oligonucleotide of SEQ ID NO:20 weekly. In yet another embodiment, the combination therapy comprises 1, 3 or 10 mg/kg intravenous infusion of anti-IL-10 hum 12G8 on the first day every three weeks and intratumoral administration of 4 mg of the CpG-C type oligonucleotide of SEQ ID NO:20 on Days 1, 8, 15, 22, 43, 50, 57 and 64. In yet another embodiment, the combination therapy comprises 1, 3 or 10 mg/kg intravenous infusion of anti-IL-10 hum 12G8 on the first day every three weeks and intratumoral administration of 4 mg of the CpG-C type oligonucleotide of SEQ ID NO:20 on the first day every week for four weeks followed by every 3 weeks. In yet another embodiment, the combination therapy comprises 1, 3 or 10 mg/kg intravenous infusion of anti-IL-10 hum 12G8 on the first day every three weeks and intratumoral administration of 4 mg of the CpG-C type oligonucleotide of SEQ ID NO:20 on the first day every week for four weeks, followed by a 3 week break, and then weekly. In yet a further embodiment, the combination therapy comprises 1, 3 or 10 mg/kg intravenous infusion of anti-IL-10 hum 12G8 on the first day every three weeks for at least four or eight cycles and intratumoral administration of 4 mg of the CpG-C type oligonucleotide of SEQ ID NO:20 on Days 1, 8, 15, 22 43, 50, 57 and 64. In some embodiments, the patient is treated with the combination therapy for at least 12 weeks, 24 weeks, e.g., eight 3-week cycles. In other embodiments, the patient is treated with 10, 11 or 12 doses of the CpG-C type oligonucleotide of SEQ ID NO:20. In some embodiments, treatment with the combination therapy continues until the patient exhibits evidence of PD or a CR. In a preferred embodiment, the anti-IL-10 antibody in the combination therapy is anti-IL-10 hum 12G8, which is administered intravenously on Day 1 at a dose of 70 mg, 210 mg or 700 mg Q3W for 7 additional doses and the CpG-C type oligonucleotide of SEQ ID NO:20 is administered intratumorally at a dose of 1.0 or 4.0 mg on Days 1, 8, 15, 22, then Q3W for 6 additional doses. In another preferred embodiment, the anti-IL-10 antibody in the combination therapy is anti-IL-10 hum 12G8, which is administered intravenously on Day 1 at a dose of 210 mg or 700 mg Q3W and the CpG-C type oligonucleotide of SEQ ID NO:20 is administered intratumorally at a dose of 1.0 or 4.0 mg on Days 1, 8, 15, 22, then Q3W. In a preferred aspect of the above embodiments, when the anti-IL-10 hum 12G8 is administered on the same day as the CpG-C type oligonucleotide, the CpG-C type oligonucleotide is administered first. In a preferred aspect of the above embodiments, the CpG-C type oligonucleotide of SEQ ID NO:20 is an oligodeoxynucleotide and has a phosphothioate backbone. In a further preferred aspect of the above embodiments, the CpG-C type oligonucleotide is a sodium salt of SEQ ID NO:20 that is an oligodeoxynucleotide with a phosphothioate backbone.

The present invention also provides a medicament which comprises an anti-IL-10 antibody as described above and a pharmaceutically acceptable excipient. When the anti-IL-10 antibody is a biotherapeutic agent, e.g., a mAb, the antibody may be produced in CHO cells using conventional cell culture and recovery/purification technologies. The anti-IL-10 antibody may be lyophilized in a buffer and reconstituted for intravenous injection. The present invention also provides a medicament which comprises a TLR9 agonist and a pharmaceutically acceptable excipient, wherein the TLR9 agonist is a CpG-C type oligonucleotide. The CpG-C type oligonucleotide may be reconstituted in a physiological buffer for intratumoral injection.

The medicaments described herein may be provided as a kit which comprises a first container and a second container and a package insert. The first container contains at least one dose of a medicament comprising an anti-IL-10 antibody, the second container contains at least one dose of a medicament comprising a CpG-C type oligonucleotide, and the package insert, or label, which comprises instructions for treating a patient for cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes.

In some embodiments of the above treatment method, medicaments and uses of the invention, the individual is a human and the cancer is a solid tumor and in some embodiments, the solid tumor is bladder cancer, breast cancer, clear cell kidney cancer, squamous cell carcinoma of head and neck, lung squamous cell carcinoma, malignant melanoma, non-small-cell lung cancer (NSCLC), ovarian cancer, pancreatic cancer, prostate cancer, renal cell cancer (RCC), small-cell lung cancer (SCLC) or triple negative breast cancer. In some embodiments, the cancer is NSCLC, endometrial cancer, urothelial cancer, squamous cell carcinoma of head and neck or melanoma.

In other embodiments of the above treatment method, medicaments and uses of the invention, the individual is a human and the cancer is a Heme malignancy and in some embodiments, the Heme malignancy is acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), cutaneous T-cell lymphoma, non-Hodgkin's lymphoma (NHL), or small lymphocytic lymphoma (SLL).

In one embodiment of the above treatment method, medicaments and uses, the individual is a human, the cancer is selected from the group consisting of melanoma, squamous cell cancer of the neck, breast cancer and non-Hodgkin's lymphoma. In another embodiment, the cancer is metastatic or unresectable melanoma, advanced squamous cell cancer of the neck, breast cancer with dermal metastasis, or indolent non-Hodgkin's lymphoma. In a further embodiment, the patient has metastatic or unresectable melanoma that has failed anti-PD1 therapy, advanced squamous cell cancer of the neck that have progressed after radiation, breast cancer with dermal metastasis, indolent non-Hodgkin's lymphoma that has failed at least one prior therapy. In yet a further embodiment, the cancer is selected from the group consisting of melanoma, head and neck cancer, breast cancer and B-cell lymphoma.

In one embodiment of the above treatment method, medicaments and uses, the individual is a human, the cancer is selected from the group consisting of renal cell carcinoma, non-small cell lung cancer, bladder cancer and colorectal cancer.

These and other aspects of the invention, including the exemplary specific embodiments listed below, will be apparent from the teachings contained herein.

### Exemplary Specific Embodiments of the Invention

1. A method for treating cancer in an individual comprising administering to the individual a combination therapy which comprises an anti-IL-10 antibody or antigen-binding fragment thereof and a TLR9 agonist, wherein the TLR9 agonist is a CpG-C type oligonucleotide.
2. The method of embodiment 1, wherein the anti-IL-10 antibody is a monoclonal antibody.
3. A medicament comprising an anti-IL-10 antibody or antigen-binding fragment thereof for use in combination with a TLR9 agonist for treating cancer in an individual, wherein the anti-IL-10 antibody is a monoclonal antibody, or an antigen binding fragment thereof and the TLR9 agonist is a CpG-C type oligonucleotide.
4. A medicament comprising a TLR9 agonist for use in combination with an anti-IL-10 antibody or antigen-binding fragment thereof for treating cancer in an individual, wherein the TLR9 agonist is a CpG-C type oligonucleotide.
5. The medicament of embodiment 3 or 4, which further comprises a pharmaceutically acceptable excipient.
6. Use of an anti-IL-10 antibody or antigen-binding fragment thereof in the manufacture of a medicament for treating cancer in an individual when administered in combination with a TLR9 agonist, wherein the TLR9 agonist is a CpG-C type oligonucleotide.
7. Use of a TLR9 agonist in the manufacture of a medicament for treating cancer in an individual when administered in combination with an anti-IL-10 antibody or antigen-binding fragment thereof, wherein the TLR9 agonist is a CpG-C type oligonucleotide.
8. Use of an anti-IL-10 antibody or antigen-binding fragment thereof and a TLR9 agonist in the manufacture of medicaments for treating cancer in an individual, wherein the TLR9 agonist is a CpG-C type oligonucleotide.
9. A kit which comprises a first container, a second container and a package insert, wherein the first container comprises at least one dose of a medicament comprising an anti-IL-10 antibody or antigen-binding fragment thereof, the second container comprises at least one dose of a medicament comprising a TLR9 agonist, and the package insert comprises instructions for treating an individual for cancer using the medicaments, wherein the TLR9 agonist is a CpG-C type oligonucleotide.
10. The method, medicament, use or kit of any one of embodiments 1-9, wherein the anti-IL-10 antibody or antigen-binding fragment thereof comprises the heavy chain and light chain variable regions of SEQ ID NO:11 and SEQ ID NO:12.
11. The method, medicament, use or kit of any one of embodiments 1-9, wherein the anti-IL-10 antibody, or antigen binding fragment thereof, comprises: (a) light chain CDRs of SEQ ID NOs: 5, 6 and 7 and heavy chain CDRs of SEQ ID NOs: 8, 9 and 10.
12. The method, medicament, use or kit of any one of embodiments 1-9, wherein the anti-IL-10 antibody is an anti-IL-10 monoclonal antibody which comprises a heavy chain and a light chain, and wherein the heavy chain comprises SEQ ID NO:1 and the light chain comprises SEQ ID NO:2.
13. The method, medicament, use or kit of any one of embodiments 1-9, wherein the anti-IL-10 antibody is anti-IL-10 hum 12G8, or an anti-IL-10 hum 12G8 variant.
14. The method, medicament, use or kit of any one of embodiments 1-9, wherein the anti-IL-10 antibody, or antigen binding fragment thereof, comprises: (a) light chain CDRs of SEQ ID NOs: 31, 32 and 33 and heavy chain CDRs of SEQ ID NOs: 34, 35 and 36.
15. The method, medicament, use or kit of any one of embodiments 1-9, wherein the anti-IL-10 antibody is an anti-IL-10 monoclonal antibody which comprises a heavy chain and a light chain, and wherein the heavy chain comprises SEQ ID NO:29 and the light chain comprises SEQ ID NO:30.
16. The method, medicament, use or kit of any one of embodiments 1-9, wherein the anti-IL-10 antibody is an anti-IL-10 monoclonal antibody which comprises a heavy chain and a light chain variable region, and wherein the heavy chain variable region comprises SEQ ID NO:4 and the light chain variable region comprises SEQ ID NO:3.
17. The method, medicament, use or kit of any one of embodiments 1-16, wherein the CpG-C type oligonucleotide consists of: (a) 5′-Nₓ(TCG(N))_{y}N_{w}(X₁X₂CGX₂′X₁′(CG)ₚ)_{z,}Nᵥ (SEQ ID NO:13) wherein N are nucleosides, x=0, 1, 2 or 3, y=1, 2, 3 or 4, w=0, 1 or 2, p=0 or 1, q=0, 1 or 2, v=0 to 89 and z=1 to 20, X₁ and X₁′ are self-complementary nucleosides, and X₂ and X₂′ are self-complementary nucleosides; and (b) a palindromic sequence at least 8 bases in length wherein the palindromic sequence comprises the first (X₁X₂CGX₂′X₁′) of the (X₁X₂CGX₂′X₁′(CG)ₚ)_{z} sequences, wherein the oligonucleotide is from 12 to 100 bases in length.
18. The method, medicament, use or kit of embodiment 17, x=0, y=1, w=0, p=0 or 1, q=0, 1 or 2, v=0 to 20 and z=1, 2, 3 or 4.
19. The method, medicament, use or kit of any one of embodiments 1-16, wherein the CpG-C type oligonucleotide consists of TCGN(X₁X₂CGX₂′X₁′CG)_{z}Nᵥ (SEQ ID NO:14), wherein N are nucleosides, q=0, 1, 2, 3, 4, or 5, v=0 to 20, z=1 to 4, X₁ and X₁′ are self-complementary nucleosides, X₂ and X₂′ are self-complementary nucleosides, and wherein the oligonucleotide is at least 12 bases in length.
20. The method, medicament, use or kit of any one of embodiments 1-16, wherein the CpG-C type oligonucleotide consists of 5′-TCGN_{q}TTCGAACGTTCGAACGTTNₛ-3′ (SEQ ID NO:15), wherein N are nucleosides, q=0, 1, 2, 3, 4 or 5, s=0 to 20, and wherein the oligonucleotide is at least 12 bases in length.
21. The method, medicament, use or kit of any one of embodiments 1-16, wherein the CpG-C type oligonucleotide is selected from the group consisting of:

(SEQ ID NO: 16)
5′-TCGTCGAACGTTCGAGATGAT-3′;
(SEQ ID NO: 17)
5′-TCGTTCGAACGTTCGAACGTTCGAA-3′;
(SEQ ID NO: 18)
5′-TCGAACGTTCGAACGTTCGAACGTT-3′;
(SEQ ID NO: 19)
5′-TCGAACGTTCGAACGTTCGAATTTT-3′;
(SEQ ID NO: 20)
5′-TCGAACGTTCGAACGTTCGAACGTTCGAAT-3′;
(SEQ ID NO: 21)
5′-TCGTAACGTTCGAACGTTCGAACGTTA-3′ ;
(SEQ ID NO: 22)
5′-TCGTAACGTTCGAACGTTCGAACGTT-3′;
(SEQ ID NO: 23)
5′-TCGTAACGTTCGAACGTTCGAACGT-3′;
(SEQ ID NO: 24)
5′-TCGTAACGTTCGAACGTTCGAACG-3′;
(SEQ ID NO: 25)
5′-TCGTAACGTTCGAACGTTCGAAC-3′;
and
(SEQ ID NO: 26)
5′-TCGTAACGTTCGAACGTTCGAA-3′.

22. The method, medicament, use or kit of any one of embodiments 1-16, wherein the CpG-C type oligonucleotide has the sequence consisting of 5′-TCGAACGTTCGAACGTTCGAACGTTCGAAT-3′ (SEQ ID NO:20).
23. The method, medicament, use or kit of any one of embodiments 1-9, wherein the CpG-C type oligonucleotide has a sequence that consists of 5′-TCGTTCGAACGTTCGAACGTTCGAA-3′ (SEQ ID NO:17).
24. The method, medicament, use or kit of any one of embodiments 1-9, wherein the CpG-C type oligonucleotide is a sodium salt with the sequence of SEQ ID NO:17, and the oligonucleotide is an oligodeoxynucelotide with a phosphorothioate backbone.
25. The method, medicament, use or kit of any one of embodiments 1-9, wherein the CpG-C type oligonucleotide is a sodium salt with the sequence of SEQ ID NO:17, and the oligonucleotide is an oligodeoxynucelotide with a phosphorothioate backbone.
26. A method for treating a human individual diagnosed with cancer, comprising administering to the individual a CpG-C type oligonucleotide of SEQ ID NO:20 intratumorally at a dose of from 1 to 16 mg weekly, and anti-IL-10 hum 12G8 intravenously at a dose of from 1 to 10 mg/kg once every three weeks, preferably administering a CpG-C type oligonucleotide of SEQ ID NO:20 intratumorally at a dose of 1, 2, 4, 8 or 16 mg weekly, and anti-IL-10 hum 12G8 intravenously at a dose of 1, 3 or 10 mg/kg once every three weeks.
27. A medicament comprising anti-IL-10 hum 12G8 for use in combination with a CpG-C type oligonucleotide of SEQ ID NO:20 for treating cancer in a human individual, wherein the CpG-C type oligonucleotide of SEQ ID NO:20 is intratumorally administered to the individual at a dose of from 1 to 16 mg weekly, and anti-IL-10 hum 12G8 is intravenously administered at a dose of from 1 to 10 mg/kg once every three weeks, preferably wherein the CpG-C type oligonucleotide of SEQ ID NO:20 is intratumorally administered to the individual at a dose of 1, 2, 4, 8 or 16 mg weekly, and anti-IL-10 hum 12G8 is intravenously administered at a dose of 1, 3 or 10 mg/kg once every three weeks.
28. A medicament comprising anti-IL-10 hum 12G8 for use in combination with a CpG-C type oligonucleotide of SEQ ID NO:20 for treating cancer in a human individual, wherein the CpG-C type oligonucleotide of SEQ ID NO:20 is intratumorally administered to the individual at a dose of from 1 to 16 mg weekly for four weeks followed by once every three weeks, and anti-IL-10 hum 12G8 is intravenously administered at a dose of from 1 to 10 mg/kg once every three weeks.
29. A medicament comprising anti-IL-10 hum 12G8 for use in combination with a CpG-C type oligonucleotide of SEQ ID NO:20 for treating cancer in a human individual, wherein the anti-IL-10 hum 12G8 is administered intravenously on Day 1 at a dose of 70 mg, 210 mg or 700 mg Q3W and the CpG-C type oligonucleotide of SEQ ID NO:20 is administered intratumorally at a dose of 1.0 or 4.0 mg on Days 1, 8, 15, 22, then Q3W.
30. The method, medicament, use or kit of any of embodiments 1-29, wherein the cancer is a solid tumor.
31. The method, medicament, use or kit of any of embodiments 1-29, wherein the cancer is bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell cancer, small-cell lung cancer (SCLC) or triple negative breast cancer.
32. The method, medicament, use or kit of any of embodiments 1-29, wherein the cancer is NSCLC, RCC, endometrial cancer, urothelial cancer, squamous cell carcinoma of head and neck or melanoma.
33. The method, medicament, use or kit of any of embodiments 1-29, wherein the cancer is acute lymphoblastic leukemia (ALL), acute myeloid leukemia (AML), chronic lymphocytic leukemia (CLL), chronic myeloid leukemia (CIVIL), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), cutaneous T-cell lymphoma, or small lymphocytic lymphoma (SLL).
34. The method, medicament, use or kit of any of embodiments 1-29, wherein the cancer is melanoma, squamous cell cancer of the head and neck, breast cancer or B-cell lymphoma.
35. The method, medicament, use or kit of any of embodiments 1-29, wherein the cancer is metastatic or unresectable melanoma, advanced squamous cell cancer of the neck, breast cancer with dermal metastasis, or indolent non-Hodgkin's lymphoma.
36. The method, medicament, use or kit of any of embodiments 1-29, wherein the cancer is metastatic or unresectable melanoma that has failed anti-PD1 or anti-CTLA-4 therapy, advanced squamous cell cancer of the neck that have progressed after radiation, breast cancer with dermal metastasis, indolent non-Hodgkin's lymphoma that has failed at least one prior therapy.
37. The method, medicament, use or kit of any one of embodiments 1-29, wherein the cancer is selected from the group consisting of renal cell carcinoma, non-small cell lung cancer, bladder cancer and colorectal cancer.
38. The method, medicament, use or kit of any one of embodiments 1-16 and 26-37, wherein the CpG-C type oligonucleotide is a sodium salt with the sequence of SEQ ID NO:20, and the oligonucleotide is an oligodeoxynucelotide with a phosphorothioate backbone.
39. The method, medicament, use or kit of any one of embodiments 1-16 and 26-37, wherein the CpG-C type oligonucleotide is the sequence of SEQ ID NO:20, and the oligonucleotide is an oligodeoxynucelotide with a phosphorothioate backbone.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992)J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fuse with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998)J. Immunol. 160:3891-3898; Hsing and Bishop (1999)J. Immunol. 162:2804-2811; Everts et al. (2002)J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### EXAMPLES

### Example 1: Immunomodulation of Human Cells by C59-08

C59-08 is a sodium salt of oligodeoxynucleotide 5′-TCGAACGTTCGAACGTTCGAACGTTCGAAT-3′ (SEQ ID NO: 20) with a phosphorothioate backbone and 5′OH and 3′OH.

Human peripheral blood mononuclear cells (PBMCs) were isolated from buffy coats from two donors with Ficoll-Paque™ PLUS (GE Healthcare Bio-Sciences, Pittsburgh, Pa.) using standard Ficoll separation methods. Isolated PBMCs were washed twice in phosphate buffered saline (PBS) containing 2% fetal bovine serum (FBS), and 2 mM ethylenediaminetetraacetic acid (EDTA). The cells were resuspended and cultured in 96-well U-bottom plates at 1×10⁶ cells per well in RPMI 1640 containing 10% FBS, 2 mM L-glutamine, 100 U/mL pencillin and 100 μg/mL streptomycin. The cells were cultured in the presence of C59-08 at doses ranging from 0.016 μM to 5 μM or 7 μM control ODN 1040 in a humidifed incubator at 37° C., 5% CO₂ in final volume of 0.2 mL for 48 hours. Supernatants were harvested and assayed for IFNα2a and IL-10 using Meso Scale Discovery human IFNα2a and human IL-10 tissue culture kits (Rockville, Md.).

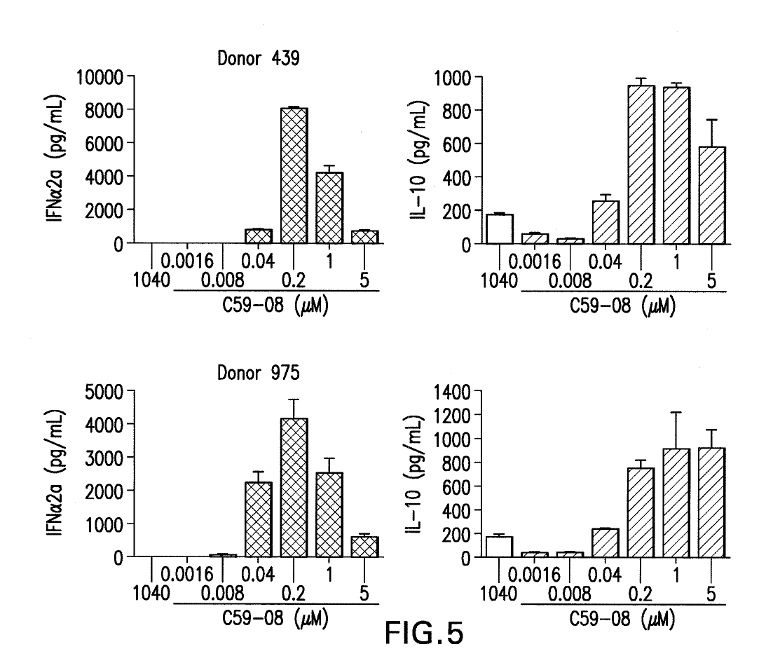

The results are shown in FIG. 5. C59-08 induces both IFNα2a and IL-10 production in human PBMCs with optimal concentration at 0.2 μM.

### Example 2: Immunomodulation of Human Tumor Specimens by C59-08 Human Tumor Histocultures

Human tumor specimens from patients were obtained from commercial sources (Bio-Options, Folio, Coversant Bio, and Boston BioSource) and University of Rochester.

Fresh tumor tissues were collected within 1 hour following surgery and placed into AQIX transportation media (AQIX, UK). Tissues were transported overnight at 4° C. to Merck Research Laboratories, Palo Alto, Calif.

The tumors were embedded in UltraPure™ low melting point agarose (Invitrogen, Carlsbad, Calif.) and were cut 400 μm with Mcllwain™ Tissue Chopper (Stoelting Co., Wood Dale, Ill.). The tumor slices were first set on the Millicell-CM cell culture insert (Millipore, Billerica, Calif.) and cultured at the interface between air and medium of 1 ml DMEM supplemented with 4.5 g/L glucose, L-glutamine, sodium pyruvate (Mediatech, Inc., Manassas, Va.), 10% FBS (SAFC Biosciences, Lenexa, Kans.), 100 U/ml penicillin, and 100 ug/ml streptomycin in humidifed incubator at 37° C., 5% CO₂.

The tumor slices were cultured in the presence of 0.1, 0.5, and 1 μM C59-08 or 1 μM control ODN 1040 for 24 hours. The tumor samples were snap frozen in dry ice and stored at 37° C. prior to processing.

### RNA Isolation and Real-Time Quantitative PCR

Total RNA was isolated by homogenization into RNA STAT-60 (Tel-Test, Friendswood, Tex.) using a polytron homogenizer. The total RNA was extracted according to the manufacturer's protocol. After precipitation with isopropanol, total RNA was re-extracted with phenol:chloroform:isoamyl alcohol (25:24:1) (Sigma-Aldrich, St. Louis, Mo.) using phase-lock light tubes.

DNase-treated total RNA was reverse-transcribed using QuantiTect Reverse Transcription (Qiagen, Valencia, Calif.) according to manufacturer's protocol. Primers were obtained commercially from Life Technologies (Foster City, Calif.). Real-time quantitative PCR on 10 ng of cDNA from each sample was performed using unlabeled primers at 900 nM each with 250 nM of FAM-labeled probe in a TAQMAN™ RTqPCR reaction on the Fluidigm Biomark sequence detection system (Fluidigm, Foster City, Calif.). Levels of ubiquitin were measured in a separate reaction and were used to normalize the data by the Δ-Δ Ct method. Using the mean cycle threshold (Ct) value for ubiquitin and the gene of interest for each sample, the following equation was used to obtain the normalized values: 1.8^{(Ct ubiquitin-Ct gene of interest)}×10⁴.

### Treatment Results

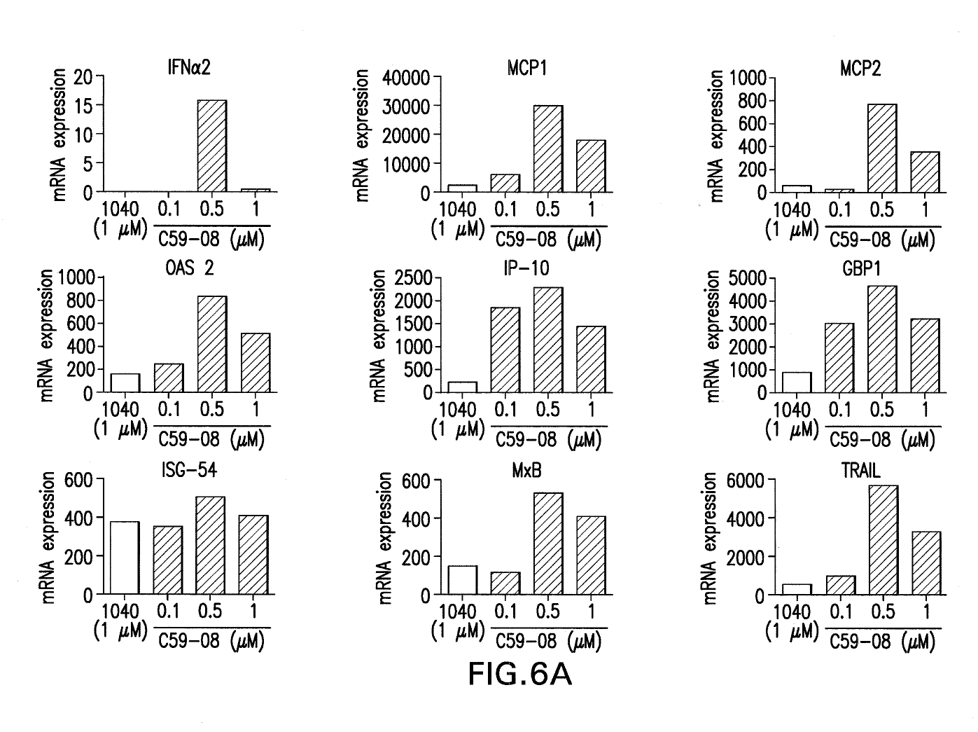

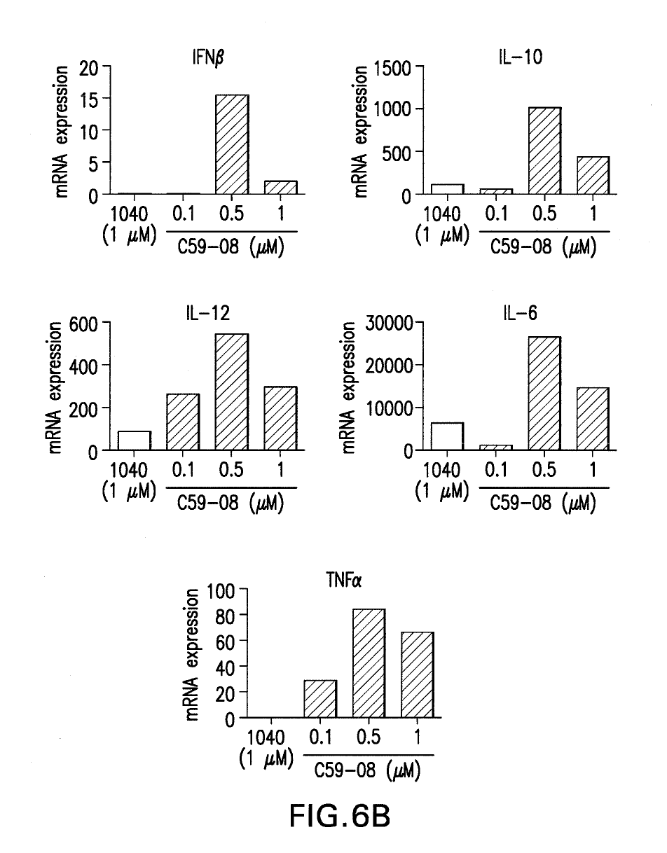

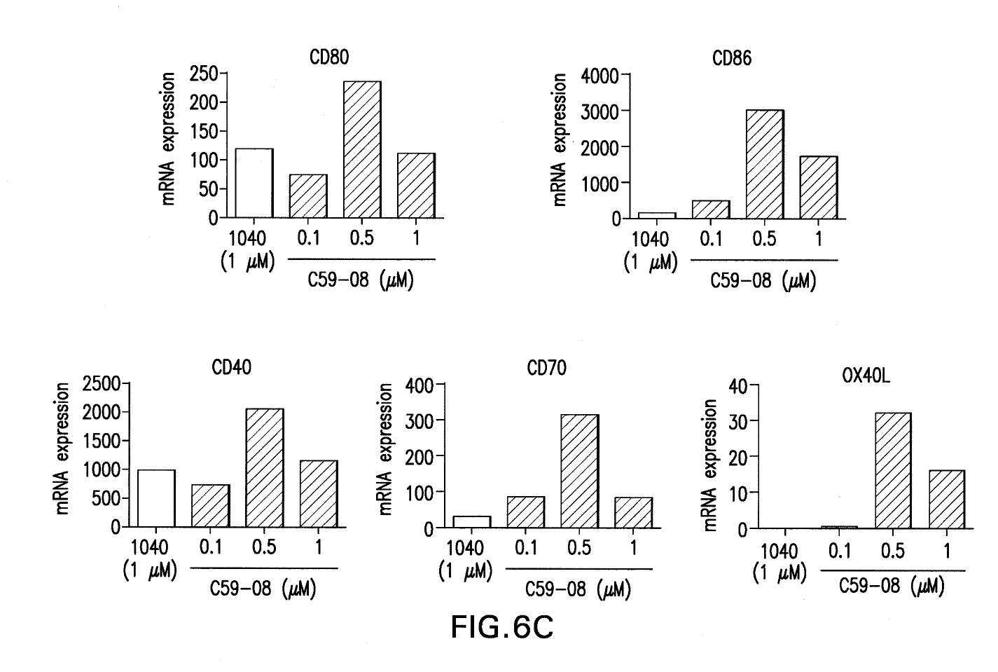

Ex vivo treatment of human tumors with C59-08 induced IFNα-inducible genes (IFNα2, MCP1, MCP2, OAS2, IP-10, GBP1, ISG-54, MxB, and TRAIL), cytokines (IFNβ, IL-10, IL-12, IL-6, and TNFα), and immune activation markers (CD80, CD86, CD40, CD70 and OX40L) in renal cell carcinoma (RC) (n=5), non-small cell lung cancer (NSCLC) (n=3), and bladder (n=1) and colorectal (n=1) cancer histocultures. Representative data with specimen from a RCC donor is shown in FIG. 6: (A) IFNα-inducible genes; (B) cytokines; and (C) immune activation markers.

### Example 3: Anti-Tumor Activity of Combination of Anti-IL-10 and Intratumoral C59-08 in Animal Model

TC40.11D8 is a mouse IgG1/kappa monoclonal antibody targeted against mouse IL-10. The mouse IgG1 isotype control is a mouse monoclonal antibody specific for adenoviral hexon 25. Both antibodies were obtained from internal sources as frozen (−80° C.) stocks.

### Formulations of Antibodies

The formulation buffer is specific for each antibody to stabilize proteins and prevent precipitation. The formulations for both TC40.11D8 and mouse IgG1 isotype control were 75 mM sodium chloride, 10 mM sodium phosphate, 3% sucrose, pH7.3.

### Oligodeoxynucleotides

Cytidine phospho-guanosine (CpG)-based phosphorothioate oligodeoxynucleotide (ODN) CpG 1826 5′-tccatgacgttcctgacgtt-3′ (SEQ ID NO: 27) (InvivoGen, San Diego, Calif.) is a mouse TLR9 specific agonist. CpG 1826 has CpG class B type sequence. CpG-based phosphorothioate ODN C59-08 (Dynavax, Berkeley, Calif.) is an agonist that activates both human and mouse TLR9. C59-08 has CpG class C type sequence. Control ODN (5′-TGA CTG TGA ACC TTA GAG ATG A-3′ (SEQ ID NO: 37) (Dynavax, Berkeley, Calif.) has a non-CpG sequence with phosphorothioate backbone.

### Formulations of Oligodeoxynucleotides

CpG 1826 was reconstituted in 0.9% sodium chloride at a concentration of 2 mg/mL, aliquoted, and stored at −20° C. C59-08 was reconstituted in phosphate buffered saline (PBS) at a concentration of 4.53 mg/mL, aliquoted, and stored at −20° C. Control ODN was reconstituted in PBS at a concentration of 4.47 mg/mL, aliquoted, and stored at −20° C.

### Animals

Approximately seven to eight week old female C57BL/6J mice were obtained from Jackson Laboratory (Sacramento, Calif.). Conventional animal chow and water were provided ad libitum. Animals were housed for one week prior to the start of the study. The average weight of the animals at the start of the study (i.e. tumor implantation) was 19 grams.

Procedures involving the care and use of animals in the study were reviewed and approved by the Institutional Animal Care and Use Committee at Merck Research Laboratories. During the study, the care and use of animals were conducted in accordance with the principles outlined in the guidance of the Association for Assessment and Accreditation of Laboratory Animal Care (AAALAC), the Animal Welfare Act, the American Veterinary Medical Association (AVMA) Euthanasia Panel on Euthanasia, and the Institute for Laboratory Animal Research (ILAR) Guide to the Care and Use of Laboratory Animals.

### Tumor Cell Line Preparation and Implantation

The TC-1 cell line, provided by Johns Hopkins University (Baltimore, Md.) is derived from mouse primary lung epithelial cells that were cotransformed with human papilloma virus (HPV-16) E6 and E7 and c-Ha.ras oncogene (Lin K Y et al. Cancer Res. 1996 Jan. 1, 56(1):21-6). TC-1 cells are syngeneic to C57BL6/J strain.

The TC-1 cells were cultured in DMEM supplemented with 10% fetal bovine serum and 0.4 mg/mL Geneticin. Sub-confluent TC-1 cells were injected subcutaneously (SC) in 0.1 mL of serum-free DMEM in both lower dorsal flanks (1×10⁵ in right flank and 0.5×10⁵ in left flank) of each animal. Animals were first shaved with electronic clippers in the areas that were used for the implantation.

### Tumor Measurements and Body Weights

Tumors were measured the day before the first dose and twice a week thereafter. Tumor length and width were measured using electronic calipers and tumor volume determined using the formula Volume (mm³)=0.5×Length×Width where length is the longer dimension. Animals were weighed the day before the first dose and twice a week thereafter. To prevent bias, any outliers by weight or tumor volume were removed and the remaining mice were grouped into various treatment groups based on the tumor volume in the right flank (referred to as injected tumor).

### Dosing Solution Preparation

Frozen stocks of the antibodies were thawed and transferred to wet ice. To avoid repeated freeze thaw, each vial of stock was thawed once and aliquots made in volumes sufficient for one time use. Polypropylene, low adhesion tubes were used for this purpose. The aliquots were stored at −80° C. Before each dosing, one aliquot was thawed and diluted to nominal concentration in the appropriate diluent. Before each dosing, aliquots of the ODNs (control ODN, CpG 1826, and C59-08) were thawed and diluted to nominal concentration in 0.9% sodium chloride.

### Administration of Antibodies and Oligodeoxynucleotides

Isotype control mIgG1 and anti-IL-10 mIgG1 were administered intraperitoneally (IP) at 10 mg/kg on Days 0, 4, 8, and 12. Control ODN (2.5 mg/kg), CpG 1826 (1 mg/kg), and C59-08 (2.5 mg/kg) were administered intratumoral (IT) only in right tumors on Days 0, 4, 8, and 12.

### Statistical Methods

Tumor volumes were compared between treatments at each day of follow-up. Follow-up of individual animals could be terminated early because of excessive tumor burden or other reasons. Depending on the reason and tumor size at the last measurement, the last observed tumor volume was treated as a lower bound on volume at all later days for that animal (right-censored data).

To compare two treatment groups on a given day, a generalization of the nonparametric Mann-Whitney (or Wilcoxon rank sum) test that allows for right-censored data was used: the Peto and Peto version of the Gehan-Breslow test. Two-sided p-values were estimated from 20,000 random reassignments of animals between the two treatments being compared. To control the familywise error rate across all time points for a given pair of treatments, p-values were multiplicity adjusted by applying the maxT procedure of Westfall and Young to the permutation distributions. A p-value of less than 0.05 was used to define statistical significance.

For descriptive purposes, volumes for each day and treatment group were summarized by their median. To allow for censoring, a distribution function for each day and treatment group was estimated by the Kaplan-Meier method, with confidence band using Greenwood's formula on a log scale. The median was estimated as the 50th percentile of the distribution function, with confidence interval obtained by inverting the confidence band. A 68% confidence level was used, to be comparable to the common “mean±SE” format for summarizing data, since the latter is approximately a 68% confidence interval for the mean.

When follow-up of an animal was terminated early, the reason was categorized and the animal's data were handled as follows: (1) tumor burden: right-censor at last measured value; (2) tumor ulceration: right-censor at last measured value, provided this exceeded a threshold (1000 mm³); otherwise omit animal at later times; (3) weight loss/ill (including found dead with evidence of illness): omit animal at later times; and (4) unrelated to treatment (e.g., accident found dead with no evidence of illness, administrative termination): right-censor at last measured value, provided this exceeded a threshold (1000 mm³); otherwise omit animal at later times.

### Treatment Results

TC-1 tumor-bearing C57BL/6J mice were grouped into 5 treatment groups the day before the first dose when the mean volume of tumors on right flank reached approximately 60 mm³ (39 mm³-87 mm³): (1) mIgG1 isotype control+control ODN; (2) mIgG1 isotype control+C59-08; (3) anti-IL-10+CpG 1826; (4) anti-IL-10+control ODN; and (5) anti-IL-10+C59-08. The range of volumes of tumors on left was 0 mm³-113 mm³. Complete regression (CR) of a tumor was defined as the absence of a measurable tumor at the time measurement was conducted, given that a tumor was measurable on the day that animals were grouped.

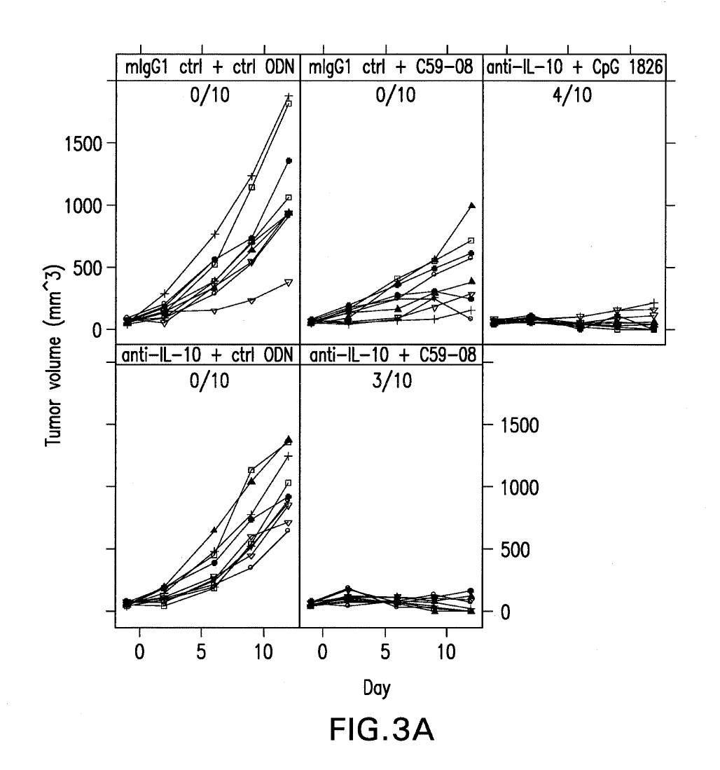

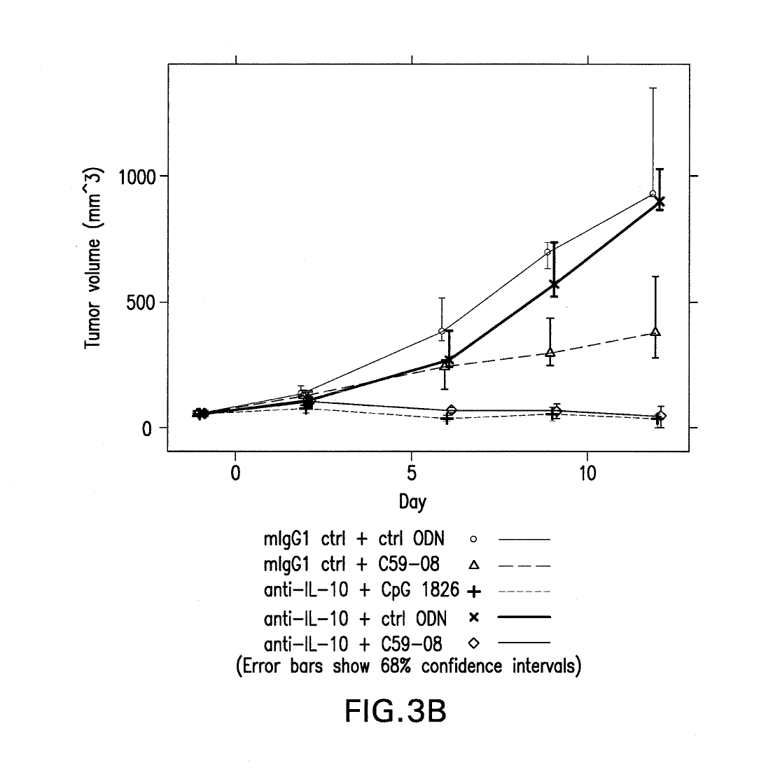

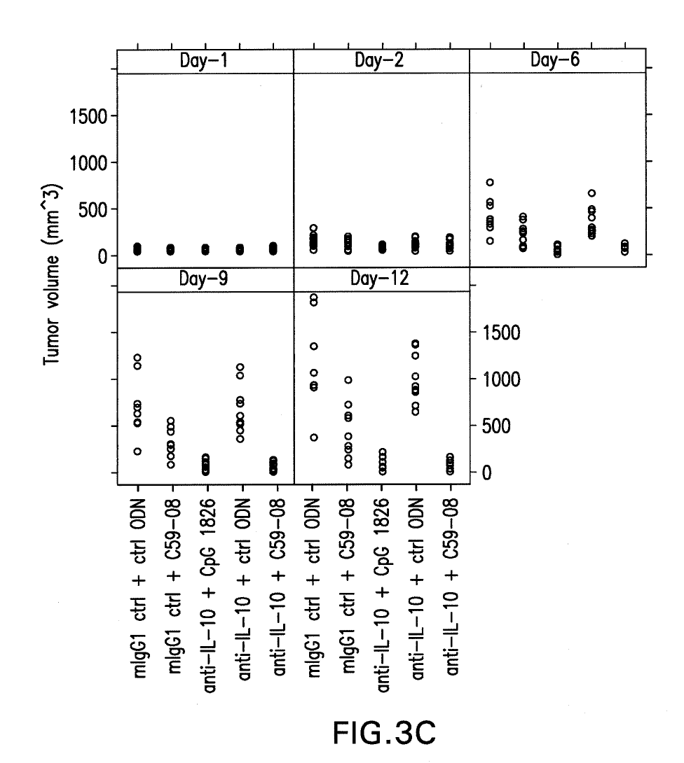

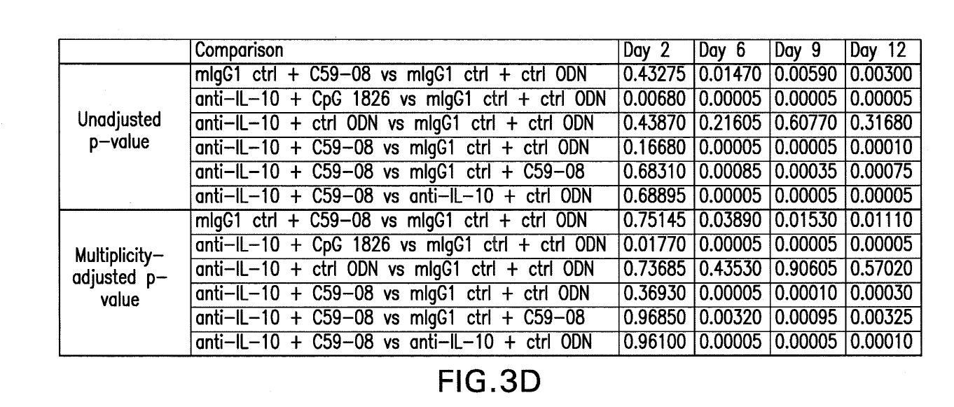

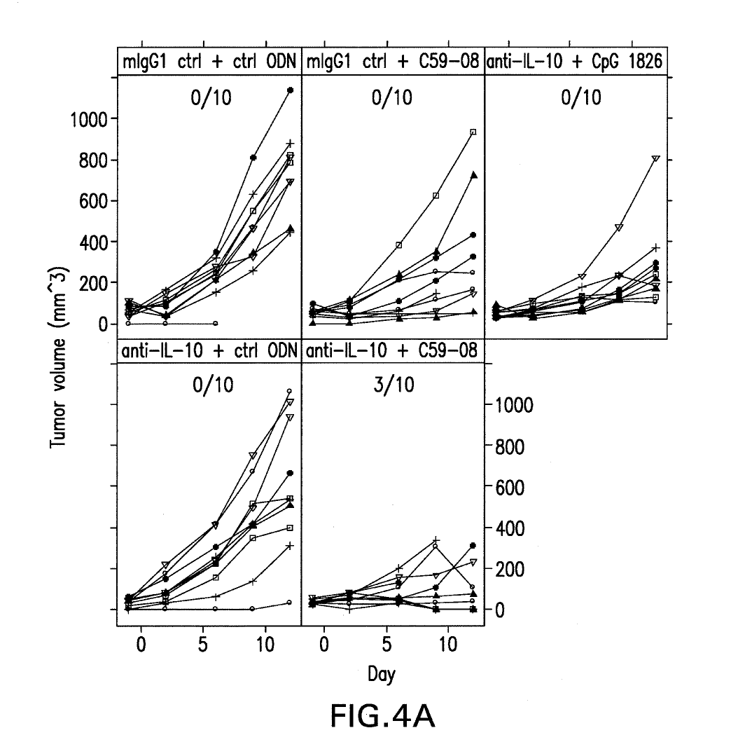

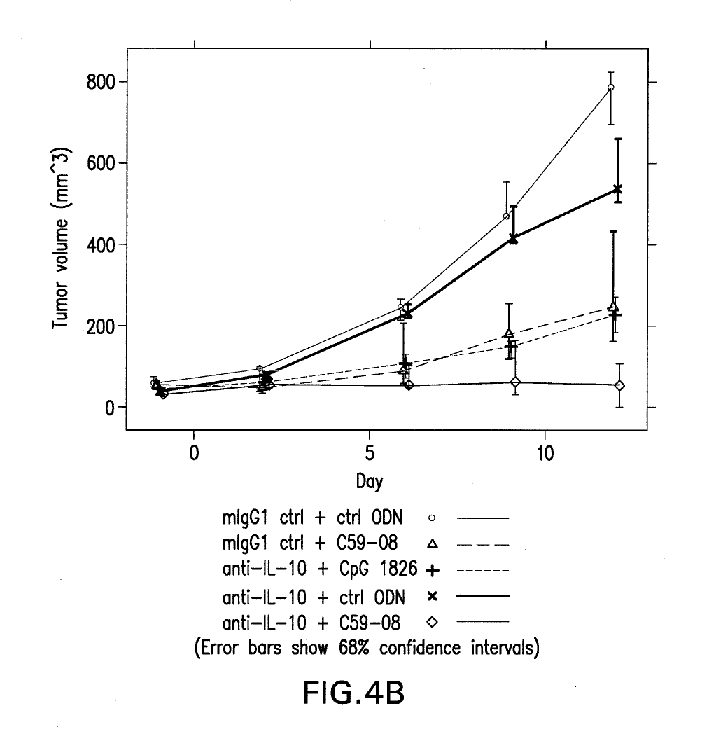

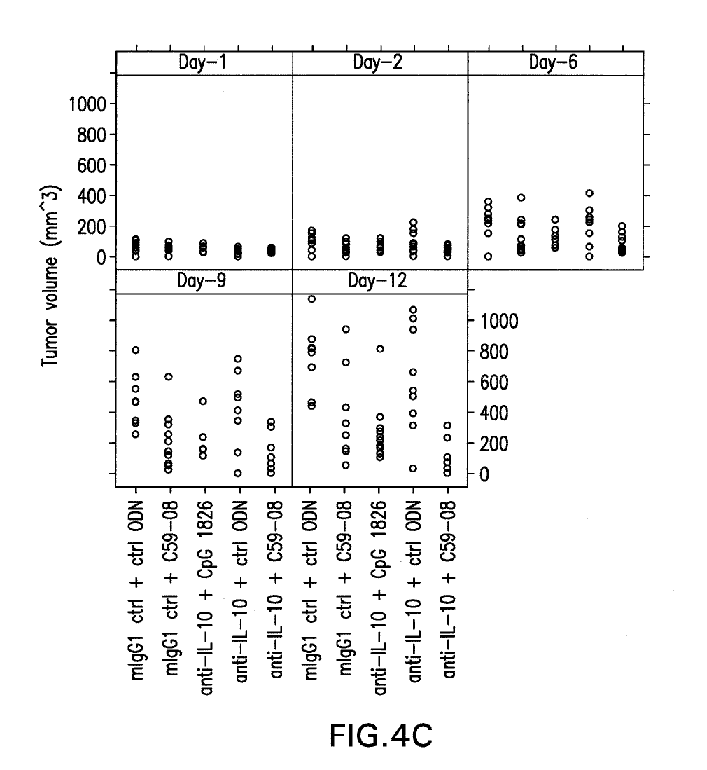

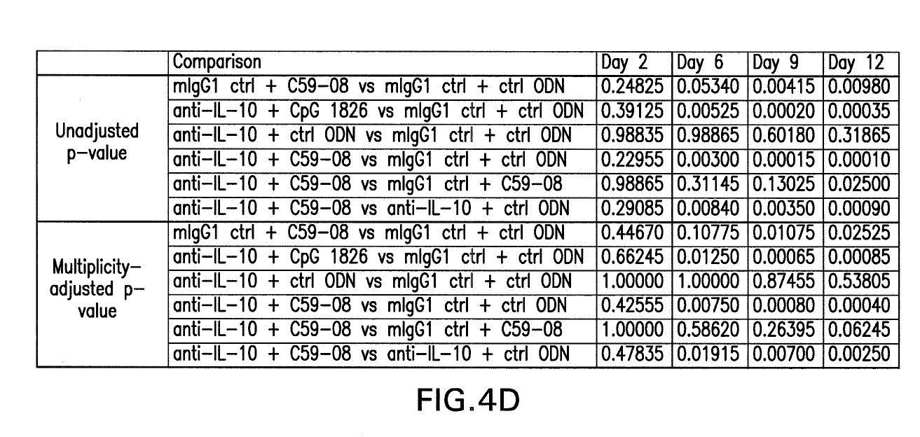

The results are shown in FIGS. 3 and 4. Anti-IL-10 in combination with either intratumoral CpG 1826 (Group 3) or C59-08 (Group 5) resulted in CRs of injected tumors in at least 3 animals (FIG. 3A). However, only anti-IL-10 in combination with C59-08 (Group 5) resulted in CRs (three of ten animals) of non-injected tumors (FIG. 4A). Other treatments including C59-C8 monotherapy (Group 2) did not result in CRs of either injected or non-injected tumors.

Compared to control treatment, anti-IL-10 monotherapy, and C59-C8 monotherapy, administration of anti-IL-10 in combination with C59-08 (IT) resulted in significantly reduced volumes of injected tumors for Days 6, 9, and 12 (p<0.05, multiplicity adjusted across time points) (FIG. 3B-D). Compared to control treatment and anti-IL-10 monotherapy, administration of anti-IL-10 in combination with C59-08 (IT) resulted in significantly reduced volumes of non-injected tumors for Days 6, 9, and 12 (p<0.05, multiplicity adjusted across time points) (FIG. 4B-D).

### Example 4: Anti-IL-10 Hum 12G8 in Combination with C59-08 in Subjects with Advanced Tumors

A phase 1b dose escalation study that tests for increasing doses of HUM12G8 in combination with dose levels of C59-08 is conducted. The study will employ the standard 3+3 design with proposed expansion cohorts at the MTD or MAD of HUM12G8. Subjects with advanced tumors with tumor present in sites accessible to injection are eligible for the study. Eligible subjects with advanced tumors will include; metastatic or unresectable melanoma that have failed anti-PD1 therapy, advanced squamous cell cancer of the head and neck that have progressed after radiation, breast cancer with dermal metastasis, indolent non-Hodgkin's lymphoma or B-cell lymphoma that has failed at least one prior therapy. All subjects in each cohort should be naïve to TLR agonist or anti-IL10 therapy.

TABLE 4
Trial Treatment(s)
Treatment during Part A: Dose finding

| | | | Dose | Route of | Regimen/Treatment |
|---|---|---|---|---|---|
| Drug | Dose/Potency | | Frequency | Administration | Period^{††} |
| Anti-IL-10 | 70 | mg | Day 1, then | Intravenous | Day 1 then Q3W for 7 |
| Hum12G8 | 210 | mg | Q3W | over 30-120 | additional doses |
| | 700 | mg | | min | |
| C59-08 | 1 | mg | Days 1, 8, | Intratumorally | Days 1, 8, 15, 22 then |
| | 4 | mg^{†} | 15, 22 then | | Q3W for 6 additional |
| | | | Q3W | | doses |

Treatment during Part B and C: Expansion cohort

| | | Dose | Route of | Regimen/Treatment |
|---|---|---|---|---|
| Drug | Dose/Potency | Frequency | Administration | Period |
| Anti-IL-10 | MTD/MAD | Every 3 | Intravenous | Day 1 then Q3W for 7 |
| Hum12G8 | | weeks | over 30-120 | additional doses |
| | | | min | |
| C59-08 | 4 mg^{†} | Days 1, 8, | Intratumorally | Days 1, 8, 15, 22 then |
| | | 15, 22 then | | Q3W for 6 additional |
| | | Q3W | | doses |

^{†}Note, if the 4 mg C59-08 and Anti-IL-10 Hum12G8 70 mg combination (cohort 2) is not tolerated, then the dose of C59-08 for subsequent cohorts (3 & 4) in part A and expansion cohorts (Parts B and C) is 1 mg.
^{††}When Hum12G8 and C59-08 are scheduled at the same time, C59-08 is administered first.

### Dose Escalation

The following three dosing cohorts were chosen with administration of the higher fixed dose of C59-08 to increase immune activation combined with escalating doses of HUM12G8. All dose levels in Part A will follow 3+3 design. Dose escalation of HUM12G8 is continued to identify a preliminary MTD or MAD. The dosing for each cohort is as follows:

1) 1 mg C59-08 intratumorally and 70 mg HUM12G8 intravenously
2) 4 mg C59-08 intratumorally and 70 mg HUM12G8 intravenously
3) 4 mg C59-08 intratumorally and 210 mg HUM12G8 intravenously
4) 4 mg C59-08 intratumorally and 700 mg HUM12G8 intravenously
If the 4 mg C59-08 and 70 mg HUM12G8 combination (cohort 2) is not tolerated, then C59-08 is de-escalated to a 1 mg dose for subsequent cohorts (3 & 4).

All references cited herein are incorporated by reference to the same extent as if each individual publication, database entry (e.g. Genbank sequences or GeneID entries), patent application, or patent, was specifically and individually indicated to be incorporated by reference. U.S. applications 62/169,321 and 62/168,470 are incorporated herein by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

1. A method for treating cancer in a human patient comprising administering to the individual a combination therapy which comprises an anti-IL-10 antibody or antigen-binding fragment thereof and a TLR9 agonist, wherein the TLR9 agonist is a CpG-C type oligonucleotide.

2. The method of claim 1, wherein the anti-IL-10 antibody is a monoclonal antibody, a humanized antibody, a chimeric antibody, or a fully human antibody.

3. The method of claim 1, wherein the anti-IL-10 antibody, or antigen binding fragment thereof, comprises: (a) light chain CDRs of SEQ ID NOs: 5, 6 and 7 (b) and heavy chain CDRs of SEQ ID NOs: 8, 9 and 10.

4. The method of claim 1, wherein the anti-IL-10 antibody or antigen-binding fragment thereof comprises the heavy chain and light chain variable regions of SEQ ID NO:11 and SEQ ID NO:12.

5. The method of claim 1, wherein the anti-IL-10 antibody or antigen binding fragment thereof is anti-IL-10 hum 12G8 or an antigen binding fragment thereof, or an anti-IL-10 hum 12G8 variant or an antigen binding fragment thereof.

6. The method of claim 1, wherein the anti-IL-10 antibody is an anti-IL-10 monoclonal antibody which comprises a heavy chain and a light chain, and wherein the heavy chain comprises SEQ ID NO:1 and the light chain comprises SEQ ID NO:2.

7. The method of claim 1, wherein the CpG-C type oligonucleotide consists of:
(a) 5′-Nₓ(TCG(N_{q}))_{y}N_{w}(X₁X₂CGX₂′X₁′(CG)ₚ)_{z,}Nᵥ (SEQ ID NO:13) wherein N are nucleosides, x=0, 1, 2 or 3, y=1, 2, 3 or 4, w=0, 1 or 2, p=0 or 1, q=0, 1 or 2, v=0 to 89 and z=1 to 20, X₁ and X₁′ are self-complementary nucleosides, and X₂ and X₂′ are self-complementary nucleosides; and
(b) a palindromic sequence at least 8 bases in length wherein the palindromic sequence comprises the first (X₁X₂CGX₂′X₁′) of the (X₁X₂CGX₂′X₁′(CG)ₚ)_{z} sequences, wherein the oligonucleotide is from 12 to 100 bases in length.

8. The method of claim 7, wherein x=0, y=1, w=0, p=0 or 1, q=0, 1 or 2, v=0 to 20 and z=1, 2, 3 or 4.

9. The method of claim 1, wherein the CpG-C type oligonucleotide consists of TCGN_{q}(X₁X₂CGX₂′X₁′CG)_{z}Nᵥ (SEQ ID NO:14), wherein N are nucleosides, q=0, 1, 2, 3, or 4, v=0 to 20, z=1 to 4, X₁ and X₁′ are self-complementary nucleosides, X₂ and X₂′ are self-complementary nucleosides, and wherein the oligonucleotide is at least 12 bases in length.

10. The method of claim 1, wherein the CpG-C type oligonucleotide consists of 5′-TCGN_{q}TTCGAACGTTCGAACGTTNₛ-3′ (SEQ ID NO:15), wherein N are nucleosides, q=0, 1, 2, 3, or 4, s=0 to 20, and wherein the oligonucleotide is at least 12 bases in length.

11. The method of claim 1, wherein the CpG-C type oligonucleotide has a sequence that consists of 5′-TCGAACGTTCGAACGTTCGAACGTTCGAAT-3′ (SEQ ID NO:20).

12. The method of claim 1, wherein the CpG-C type oligonucleotide has a sequence that consists of 5′-TCGTTCGAACGTTCGAACGTTCGAA-3′ (SEQ ID NO:17).

13. The method of claim 1, wherein the CpG-C type oligonucleotide is a sodium salt with the sequence of SEQ ID NO:17, and the oligonucleotide is an oligodeoxynucleotide with a phosphorothioate backbone.

14. A method for treating a human individual diagnosed with cancer, comprising administering to the individual a CpG-C type oligonucleotide of SEQ ID NO:20 intratumorally at a dose of from 1 to 16 mg weekly, and anti-IL-10 hum 12G8 intravenously at a dose of from 1 to 10 mg/kg once every three weeks.

15. A method for treating a human individual diagnosed with cancer, comprising administering to the individual a CpG-C type oligonucleotide of SEQ ID NO:20 intratumorally at a dose of from 1 to 16 mg weekly for four weeks followed by once every three weeks, and anti-IL-10 hum 12G8 intravenously at a dose of from 1 to 10 mg/kg once every three weeks.

16. A method for treating a human individual diagnosed with cancer, comprising administering to the individual a CpG-C type oligonucleotide of SEQ ID NO:20 intratumorally at a dose of 1.0 or 4.0 mg on Days 1, 8, 15, 22, then once every three weeks and anti-IL-10 hum 12G8 intravenously on Day 1 at a dose of 70 mg, 210 mg or 700 mg once every three weeks.

17. The method of claim 1, wherein the cancer is selected from the group consisting of melanoma, squamous cell cancer of the neck, breast cancer, and non-Hodgkin's lymphoma.

18. The method of claim 1, wherein the cancer is selected from the group consisting of melanoma, head and neck cancer, breast cancer, and B-cell lymphoma.

19. The method of claim 1, wherein the cancer is selected from the group consisting of metastatic or unresectable melanoma, advanced squamous cell cancer of the neck, breast cancer with dermal metastasis, and indolent non-Hodgkin's lymphoma.

20. The method of claim 1, wherein the cancer is selected from the group consisting of renal cell carcinoma, non-small cell lung cancer, bladder cancer, and colorectal cancer.

21. The method of claim 1, wherein the CpG-C type oligonucleotide is a sodium salt with the sequence of SEQ ID NO: 20, and the oligonucleotide is an oligodeoxynucleotide with a phosphorothioate backbone.

22. The method of claim 16, wherein the CpG-C type oligonucleotide is a sodium salt with the sequence of SEQ ID NO: 20, and the oligonucleotide is an oligodeoxynucleotide with a phosphorothioate backbone.

23. The method of claim 1, wherein the CpG-C type oligonucleotide sequence is SEQ ID NO: 20, and the oligonucleotide is an oligodeoxynucleotide with a phosphorothioate backbone.

24. The method of claim 16, wherein the CpG-C type oligonucleotide sequence is SEQ ID NO: 20, and the oligonucleotide is an oligodeoxynucleotide with a phosphorothioate backbone.


SEQUENCE LISTING (39 sequences)

SEQ ID NO: 1 - protein, 449 aa, Homo sapiens
QVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Homo sapiens
DIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 111 aa, Mus sp.
DIVLTQSPASLAVSLGQRATISCRASESVDDYGHSFMHWYQQKPGQPPKLLIWRASTLESGIPARFSGSGSRTDFTLTINPVEADDVATYYCQQGNEDPWTFGGGTKLEIK

SEQ ID NO: 4 - protein, 118 aa, Mus sp.
QVQLKQSGPGLVQPSQSLSITCTVSGFSLTNYGVHWVRQSPGKGLEWLGVIWSGGSTDYNAAFISRLSINKDNSKSQVFFKMNSLQANDTAIYYCARNRGYDVYFDYWGQGTTLTVSS

SEQ ID NO: 5 - protein, 11 aa, Rattus norvegicus
KTSQNIFENLA

SEQ ID NO: 6 - protein, 8 aa, Rattus norvegicus
YNASPLQA

SEQ ID NO: 7 - protein, 8 aa, Rattus norvegicus
HQYYSGYT

SEQ ID NO: 8 - protein, 10 aa, Rattus norvegicus
GFTFSDYHMA

SEQ ID NO: 9 - protein, 17 aa, Rattus norvegicus
SITLDATYTYYRDSVRG

SEQ ID NO: 10 - protein, 10 aa, Rattus norvegicus
HRGFSVWLDY

SEQ ID NO: 11 - protein, 120 aa, Homo sapiens
QVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSA

SEQ ID NO: 12 - protein, 111 aa, Homo sapiens
DIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKRTVAA

SEQ ID NO: 13 - DNA, 274 bp, Artificial Sequence
  Description of Artificial Sequence Synthetic polynucleotide
  modified_base (1)..(3): a, c, t or g
  misc_feature (1)..(3): This region may encompass 0-3 nucleotides wherein some positions may be absent
  misc_feature (4)..(23): This region may encompass 1-4 ′tcgnn′ repeats wherein some positions may be absent
  modified_base (7)..(8): a, c, t or g
  misc_feature (7)..(8): This region may encompass 0-2 nucleotides wherein some positions may be absent
  modified_base (12)..(13): a, c, t or g
  misc_feature (12)..(13): This region may encompass 0-2 nucleotides wherein some positions may be absent
  modified_base (17)..(18): a, c, t or g
  misc_feature (17)..(18): This region may encompass 0-2 nucleotides wherein some positions may be absent
  modified_base (22)..(23): a, c, t or g
  misc_feature (22)..(23): This region may encompass 0-2 nucleotides wherein some positions may be absent
  modified_base (24)..(25): a, c, t or g
  misc_feature (24)..(25): This region may encompass 0-2 nucleotides wherein some positions may be absent
  modified_base (26)..(27): a, c, t or g
  misc_feature (26)..(185): This region may encompass 1-20 ′nncgnncg′ repeats wherein some positions may be absent
  modified_base (30)..(31): a, c, t or g
  misc_feature (32)..(33): May or may not be present
  modified_base (34)..(35): a, c, t or g
  modified_base (38)..(39): a, c, t or g
  misc_feature (40)..(41): May or may not be present
  modified_base (42)..(43): a, c, t or g
  modified_base (46)..(47): a, c, t or g
  misc_feature (48)..(49): May or may not be present
  modified_base (50)..(51): a, c, t or g
  modified_base (54)..(55): a, c, t or g
  misc_feature (56)..(57): May or may not be present
  modified_base (58)..(59): a, c, t or g
  modified_base (62)..(63): a, c, t or g
  misc_feature (64)..(65): May or may not be present
  modified_base (66)..(67): a, c, t or g
  modified_base (70)..(71): a, c, t or g
  misc_feature (72)..(73): May or may not be present
  modified_base (74)..(75): a, c, t or g
  modified_base (78)..(79): a, c, t or g
  misc_feature (80)..(81): May or may not be present
  modified_base (82)..(83): a, c, t or g
  modified_base (86)..(87): a, c, t or g
  misc_feature (88)..(89): May or may not be present
  modified_base (90)..(91): a, c, t or g
  modified_base (94)..(95): a, c, t or g
  misc_feature (96)..(97): May or may not be present
  modified_base (98)..(99): a, c, t or g
  modified_base (102)..(103): a, c, t or g
  misc_feature (104)..(105): May or may not be present
  modified_base (106)..(107): a, c, t or g
  modified_base (110)..(111): a, c, t or g
  misc_feature (112)..(113): May or may not be present
  modified_base (114)..(115): a, c, t or g
  modified_base (118)..(119): a, c, t or g
  misc_feature (120)..(121): May or may not be present
  modified_base (122)..(123): a, c, t or g
  modified_base (126)..(127): a, c, t or g
  misc_feature (128)..(129): May or may not be present
  modified_base (130)..(131): a, c, t or g
  modified_base (134)..(135): a, c, t or g
  misc_feature (136)..(137): May or may not be present
  modified_base (138)..(139): a, c, t or g
  modified_base (142)..(143): a, c, t or g
  misc_feature (144)..(145): May or may not be present
  modified_base (146)..(147): a, c, t or g
  modified_base (150)..(151): a, c, t or g
  misc_feature (152)..(153): May or may not be present
  modified_base (154)..(155): a, c, t or g
  modified_base (158)..(159): a, c, t or g
  misc_feature (160)..(161): May or may not be present
  modified_base (162)..(163): a, c, t or g
  modified_base (166)..(167): a, c, t or g
  misc_feature (168)..(169): May or may not be present
  modified_base (170)..(171): a, c, t or g
  modified_base (174)..(175): a, c, t or g
  misc_feature (176)..(177): May or may not be present
  modified_base (178)..(179): a, c, t or g
  modified_base (182)..(183): a, c, t or g
  misc_feature (184)..(185): May or may not be present
  modified_base (186)..(274): a, c, t or g
  misc_feature (186)..(274): This region may encompass 0-89 nucleotides wherein some positions may be absent
  See specification as filed for detailed description of substitutions and preferred embodiments
nnntcgnntcgnntcgnntcgnnnnnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 14 - DNA, 60 bp, Artificial Sequence
  Description of Artificial Sequence Synthetic oligonucleotide
  modified_base (4)..(8): a, c, t or g
  misc_feature (4)..(8): This region may encompass 0-5 nucleotides wherein some positions may be absent
  misc_feature (9)..(40): This region may encompass 1-4 ′nncgnncg′ repeats wherein some positions may be absent
  modified_base (9)..(10): a, c, t or g
  modified_base (13)..(14): a, c, t or g
  modified_base (17)..(18): a, c, t or g
  modified_base (21)..(22): a, c, t or g
  modified_base (25)..(26): a, c, t or g
  modified_base (29)..(30): a, c, t or g
  modified_base (33)..(34): a, c, t or g
  modified_base (37)..(38): a, c, t or g
  modified_base (41)..(60): a, c, t or g
  misc_feature (41)..(60): This region may encompass 0-20 nucleotides wherein some positions may be absent
  See specification as filed for detailed description of substitutions and preferred embodiments
tcgnnnnnnncgnncgnncgnncgnncgnncgnncgnncgnnnnnnnnnnnnnnnnnnnn

SEQ ID NO: 15 - DNA, 46 bp, Artificial Sequence
  Description of Artificial Sequence Synthetic oligonucleotide
  modified_base (4)..(8): a, c, t or g
  misc_feature (4)..(8): This region may encompass 0-5 nucleotides wherein some positions may be absent
  modified_base (27)..(46): a, c, t or g
  misc_feature (27)..(46): This region may encompass 0-20 nucleotides wherein some positions may be absent
  See specification as filed for detailed description of substitutions and preferred embodiments
tcgnnnnnttcgaacgttcgaacgttnnnnnnnnnnnnnnnnnnnn

SEQ ID NO: 16 - DNA, 21 bp, Artificial Sequence
  Description of Artificial Sequence Synthetic construct
tcgtcgaacgttcgagatgat

SEQ ID NO: 17 - DNA, 25 bp, Artificial Sequence
  Description of Artificial Sequence Synthetic construct
tcgttcgaacgttcgaacgttcgaa

SEQ ID NO: 18 - DNA, 25 bp, Artificial Sequence
  Description of Artificial Sequence Synthetic construct
tcgaacgttcgaacgttcgaacgtt

SEQ ID NO: 19 - DNA, 25 bp, Artificial Sequence
  Description of Artificial Sequence Synthetic construct
tcgaacgttcgaacgttcgaatttt

SEQ ID NO: 20 - DNA, 30 bp, Artificial Sequence
  Description of Artificial Sequence Synthetic construct
tcgaacgttcgaacgttcgaacgttcgaat

SEQ ID NO: 21 - DNA, 27 bp, Artificial Sequence
  Description of Artificial Sequence Synthetic construct
tcgtaacgttcgaacgttcgaacgtta

SEQ ID NO: 22 - DNA, 26 bp, Artificial Sequence
  Description of Artificial Sequence Synthetic construct
tcgtaacgttcgaacgttcgaacgtt

SEQ ID NO: 23 - DNA, 25 bp, Artificial Sequence
  Description of Artificial Sequence Synthetic construct
tcgtaacgttcgaacgttcgaacgt

SEQ ID NO: 24 - DNA, 24 bp, Artificial Sequence
  Description of Artificial Sequence Synthetic construct
tcgtaacgttcgaacgttcgaacg

SEQ ID NO: 25 - DNA, 23 bp, Artificial Sequence
  Description of Artificial Sequence Synthetic construct
tcgtaacgttcgaacgttcgaac

SEQ ID NO: 26 - DNA, 22 bp, Artificial Sequence
  Description of Artificial Sequence Synthetic construct
tcgtaacgttcgaacgttcgaa

SEQ ID NO: 27 - DNA, 20 bp, Artificial Sequence
  Description of Artificial Sequence Synthetic construct
tccatgacgttcctgacgtt

SEQ ID NO: 28 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 29 - protein, 448 aa, Homo sapiens
QVQLVESGGGVVQPGRSLRLSCAASGFSLTNYGVHWVRQAPGKGLEWVAVIWSGGSTDYNAAFISRFTISRDNSKNTLYLQMNSLRAEDTAVYYCARNRGYDVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 218 aa, Homo sapiens
EIVLTQSPGTLSLSPGERATLSCRASESVDDYGHSFMHWYQQKPGQAPRLLIYRASTLESGIPDRFSGSGSGTDFTLTISRLEPEDFAVYYCQQGNEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 15 aa, Mus sp.
RASESVDDYGHSFMH

SEQ ID NO: 32 - protein, 7 aa, Mus sp.
RASTLES

SEQ ID NO: 33 - protein, 9 aa, Mus sp.
QQGNEDPWT

SEQ ID NO: 34 - protein, 10 aa, Mus sp.
GFSLTNYGVH

SEQ ID NO: 35 - protein, 16 aa, Mus sp.
VIWSGGSTDYNAAFIS

SEQ ID NO: 36 - protein, 10 aa, Mus sp.
NRGYDVYFDY

SEQ ID NO: 37 - DNA, 22 bp, Artificial Sequence
  Description of Artificial Sequence Synthetic construct
tgactgtgaaccttagagatga

SEQ ID NO: 38 - DNA, 20 bp, Artificial Sequence
  Description of Artificial Sequence Synthetic oligonucleotide
  misc_feature (1)..(20): This sequence may encompass 1-4 ′tcgnn′ repeats wherein some positions may be absent
  modified_base (4)..(5): a, c, t or g
  misc_feature (4)..(5): This region may encompass 0-2 nucleotides wherein some positions may be absent
  modified_base (9)..(10): a, c, t or g
  misc_feature (9)..(10): This region may encompass 0-2 nucleotides wherein some positions may be absent
  modified_base (14)..(15): a, c, t or g
  misc_feature (14)..(15): This region may encompass 0-2 nucleotides wherein some positions may be absent
  modified_base (19)..(20): a, c, t or g
  misc_feature (19)..(20): This region may encompass 0-2 nucleotides wherein some positions may be absent
  See specification as filed for detailed description of substitutions and preferred embodiments
tcgnntcgnntcgnntcgnn

SEQ ID NO: 39 - DNA, 32 bp, Artificial Sequence
  Description of Artificial Sequence Synthetic oligonucleotide
  misc_feature (1)..(32): This sequence may encompass 1-4 ′nncgnncg′ repeats wherein some positions may be absent
  modified_base (1)..(2): a, c, t or g
  modified_base (5)..(6): a, c, t or g
  misc_feature (7)..(8): May or may not be present
  modified_base (9)..(10): a, c, t or g
  modified_base (13)..(14): a, c, t or g
  misc_feature (15)..(16): May or may not be present
  modified_base (17)..(18): a, c, t or g
  modified_base (21)..(22): a, c, t or g
  misc_feature (23)..(24): May or may not be present
  modified_base (25)..(26): a, c, t or g
  modified_base (29)..(30): a, c, t or g
  misc_feature (31)..(32): May or may not be present
  See specification as filed for detailed description of substitutions and preferred embodiments
nncgnncgnncgnncgnncgnncgnncgnncg